(19) United States
(12) Patent Application Publication
(10) Pub. No.: US 2019/0374207 A1
(43) Pub. Date: Dec. 12, 2019

(54) INGESTIBLE DEVICE WITH ON-BOARD FLUOROMETER AND RELATED SYSTEMS AND METHODS

(71) Applicant: Progenity, Inc., San Diego, CA (US)

(72) Inventors: Mitchell Lawrence Jones, La Jolla, CA (US); Todd Louis Harris, Fremont, CA (US); John Paul Bowen, Pittsford, NY (US); Aaron Olafur Laurence Philippsen, Victoria (CA); Jeffrey A. Shimizu, Poway, CA (US); Sharat Singh, Rancho Santa Fe, CA (US)

(21) Appl. No.: 16/434,118

(22) Filed: Jun. 6, 2019

Related U.S. Application Data
(60) Provisional application No. 62/681,589, filed on Jun. 6, 2018.

(51) Int. Cl. A61B 10/00; A61B 5/00

(52) U.S. Cl. CPC A61B 10/0045; A61B 2010/0061; A61B 5/0071

(57) ABSTRACT

An ingestible device includes a sample chamber configured to hold a sample, the sample chamber including a base having an aperture; a fluorometer separated from the sample chamber by the sample chamber base, the fluorometer including (I) a light source configured to emit excitation light, (II) a light guide configured to guide the excitation light from the light source to the sample, (III) a photodetector configured to detect emission light generated via the interaction of the excitation light with the sample. The photodetector is configured to receive the emission light from the sample through the aperture. Additionally, the fluorometer includes (IV) an emission filter between the aperture and the photodetector, the emission filter configured to (i) transmit the emission light incident thereon, and (ii) block excitation light incident thereon. In addition, the ingestible device includes a housing that houses the sample chamber and the fluorometer.

### INCORPORATION BY REFERENCE

The application incorporates by reference the following co-pending U.S. patent applications: U.S. Ser. No. 14/460,893, entitled “Ingestible Medical Device,” and filed Aug. 15, 2014; U.S. Ser. No. 15/514,413, entitled “Electromechanical Pill Device with Localization Capabilities,” and filed Mar. 24, 2017; U.S. Ser. No. 15/680,400, entitled “Systems and Methods for Obtaining Samples using Ingestible Devices,” filed on Aug. 18, 2017; U.S. Ser. No. 15/680,430, entitled “Sampling Systems and Related Materials and Methods,” filed on Aug. 18, 2017; U.S. Ser. No. 15/699,848, entitled “Electromechanical Ingestible Delivery of a Dispensable Substance,” filed on Sep. 8, 2017; U.S. Ser. No. 15/835,270, entitled “Gastrointestinal Tract Detection Methods, Device and Systems,” and filed Dec. 7, 2017; U.S. Ser. No. 15/835,237, entitled “Gastrointestinal Tract Detection Methods, Device and Systems,” and filed Dec. 7, 2017; U.S. Ser. No. 15/835,292, entitled “Gastrointestinal Tract Detection Methods, Device and Systems,” and filed Dec. 7, 2017; U.S. Ser. No. 15/844,349, entitled “Ingestible Device and Associated Methods,” filed Dec. 15, 2017; U.S. Ser. No. 15/844,381, entitled “Ingestible Device and Associated Methods,” filed Dec. 15, 2017; U.S. Ser. No. 15/844,427, entitled “Ingestible Device and Associated Methods,” filed Dec. 15, 2017; Ser. No. 15/694,458, entitled “Systems and Methods for Extracting a Sample from an Ingestible Device,” filed on Mar. 15, 2018; U.S. Ser. No. 15/940,407, entitled “Localization Systems and Methods for an Optoelectromechanical Pill Device,” filed on Mar. 29, 2018; U.S. Ser. No. 16/299,537, entitled “Ingestible Device With Relatively Large Payload Volume,” filed Mar. 12, 2019; and PCT/US2019/034795, entitled “Devices and Systems for Gastrointestinal Microbiome Detection and Manipulation,” and filed May 31, 2019.

### TECHNICAL FIELD

This specification relates to an ingestible device that includes a fluorometer, as well as related systems and methods.

### BACKGROUND

Information about a sample within the gastrointestinal (GI) tract of a subject can be obtained based on intensity data or spectrally-selective intensity data or both for light emitted by the sample.

### SUMMARY

The disclosure provides an ingestible device with a fluorometer for obtaining sample information in situ within the GI tract of a subject. The sample information can be communicated outside the subject. Optionally, the sample information can be used to determine whether the subject has a particular condition or is a candidate for contracting a particular condition. Such information can be used to determine a course of treatment for the subject. In some embodiments, based on the information, ingestible device receives instructions to deliver one or more therapeutic agents to one or more locations with the GI tract of the subject. In certain embodiments, the information about the subject is used in a course of treatment that does not involve delivery of a therapeutic agent from the device with the fluorometer.

The ingestible device can include one or more regions that are designed to reduce undesirable background light. As an example, a portion of the ingestible device that houses components of the fluorometer may include a housing that has an exposed surface that has a relatively low reflectivity at one or more wavelengths used by the fluorometer. This can allow the ingestible device to provide relatively accurate and reliable data about one or more samples collected by the ingestible device. In some embodiments, such data can be collected comparatively quickly.

Intensity data or spectrally-selective intensity data or both or both for light emitted by a sample within the GI tract of a subject can be produced using a fluorometer included in an ingestible device disposed in the GI tract of the subject. First, the ingestible device retrieves the sample from the GI tract inside the ingestible device. Second, the on-board fluorometer excites the retrieved sample with excitation light and collects emission light emitted by the excited sample. The intensity data and/or spectrally-selective intensity data produced by the fluorometer for the collected emission light can be stored and/or processed in-situ by the ingestible device, or can be transmitted to a receiver remote from the ingestible device. The various embodiments of fluorometers described herein can be incorporated in an ingestible device to produce intensity data and spectrally-selective intensity data with sufficient sensitivity to characterize a variety of GI tract samples.

In general, one innovative aspect of the subject matter described in this specification can be embodied in an ingestible device that includes a sample chamber configured to hold a sample, the sample chamber comprising a base having an aperture; a fluorometer separated from the sample chamber by the sample chamber base, the fluorometer comprising (I) a light source configured to emit excitation light, (II) a light guide configured to guide the excitation light from the light source to the sample, (III) a photodetector configured to detect emission light generated via the interaction of the excitation light with the sample, wherein the photodetector is configured to receive the emission light from the sample through the aperture, and (IV) an emission filter between the aperture and the photodetector, the emission filter configured to (i) transmit the emission light incident thereon, and (ii) block excitation light incident thereon; and a housing that houses the sample chamber and the fluorometer.

The foregoing and other embodiments of the ingestible device can each optionally include one or more of the following features, alone or in combination. In particular, one embodiment includes all the following features in combination.

In some embodiments, the light guide can include one or more optical fibers. For instance, the light guide can include an optical-fiber bundle.

In some embodiments, the sample chamber can include an assay pad disposed adjacent to the aperture of the sample chamber base and configured to hold the sample. In some cases, the light guide can have an input end coupled with the light source and an output end coupled with the assay pad. Here, the light guide is configured to (i) receive the excitation light at the input end, (ii) guide the excitation light from the input end to the output end, and (iii) deliver the excitation light through the output end to the assay pad. For example, the light guide can be configured such that the output end is coupled to a surface of the assay pad facing the sample chamber to deliver the excitation light towards the fluorometer. As another example, the light guide can be configured such that the output end is coupled to a surface of the assay pad facing the fluorometer to deliver the excitation light towards the sample chamber.

In some embodiments, the light source can be disposed on an inner surface of the housing that is (i) part of the fluorometer, and (ii) oriented parallel to the sample-chamber base. In some cases, the light source can be disposed on the same inner surface of the housing as the photodetector. In some embodiments, the light source can be disposed on an inner surface of the housing that is (i) part of the fluorometer, and (ii) oriented orthogonal to the sample-chamber base.

In some embodiments, the photodetector can be disposed on an inner surface of the housing that is part of the fluorometer at a position where an optical axis of the aperture intersects the inner surface of the housing. In some embodiments, inner surfaces of the housing that are part of the fluorometer can include a light-absorbing material configured to absorb excitation light incident thereon.

In general, another innovative aspect of the subject matter described in this specification can be embodied in an ingestible device that includes a sample chamber having a base and comprising an assay pad configured to hold a sample, wherein the sample chamber base has an aperture, and the assay pad is disposed adjacent to the aperture; a fluorometer separated from the sample chamber by the sample-chamber base, the fluorometer comprising (I) an annular-shaped light source having an inner edge, the annular-shaped light source configured to emit excitation light at its inner edge, the annular-shaped light source being attached to a fluorometer-side surface of the sample-chamber base to deliver the excitation light to a portion of the assay pad that (i) protrudes through the aperture and (ii) is encircled by the inner edge of the annular-shaped light source, (II) a photodetector configured to detect emission light generated via the interaction of the excitation light with the sample, (III) a fluorescence-collection optic having an input end coupled to the assay pad and an output end coupled with the photodetector, the fluorescence-collection optic configured to (i) receive at the input end emission light emitted by the sample with a first divergence, and (ii) provide, at the output end to the photodetector, the emitted light with a second divergence smaller than the first divergence, and (IV) an emission filter between the output end of the fluorescence-collection optic and the photodetector, the emission filter configured to (i) transmit the emission light incident thereon, and (ii) block the excitation light incident thereon; and a housing that houses the sample chamber and the fluorometer.

The foregoing and other embodiments of the ingestible device can each optionally include one or more of the following features, alone or in combination. In particular, one embodiment includes all the following features in combination.

In some embodiments, the fluorescence-collection optic is configured as a compound parabolic concentrator (CPC). In some cases, the CPC can be a solid CPC formed from a dielectric material that is transparent to emission light, and a side surface of the CPC is shaped to reflect the emission light received at the input end through total internal reflection (TIR). In other cases, the CPC is a hollow CPC formed from a reflective material.

In some embodiments, the emission filter can be coupled at the output end of the fluorescence-collection optic, and the photodetector is spaced apart from the emission filter.

In all the above embodiments of the ingestible device, the photodetector is spaced apart from the emission filter by a separation distance in a range of 0-1.5 mm. In all the above embodiments of the ingestible device, the fluorescence-collection optic has a longitudinal size in a range of 5-6 mm.

In general, another innovative aspect of the subject matter described in this specification can be embodied in an ingestible device that includes a sample chamber configured to hold a sample, the sample chamber having a base with an aperture; a fluorometer separated from the sample chamber by the sample-chamber base, the fluorometer comprising (I) a light source configured to emit excitation light and configured to deliver the excitation light to the sample through the aperture, (II) a photodetector configured to detect emission light generated via the interaction of the excitation light with sample, wherein the photodetector is configured to receive the emission light from the sample through the aperture, and (III) an emission filter between the aperture and the photodetector, the emission filter configured to (i) transmit the emission light incident thereon, and (ii) block the excitation light incident thereon; and a housing that houses the sample chamber and the fluorometer. Inner surfaces of the housing that are part of the fluorometer include a light-absorbing material configured to absorb excitation light incident thereon.

The foregoing and other embodiments of the ingestible device can each optionally include one or more of the following features, alone or in combination. In particular, one embodiment includes all the following features in combination.

In some embodiments, a fluorometer-facing surface of the sample-chamber base can include the same or another light-absorbing material as the one in the light-absorbing material of the inner surfaces of the housing configured to absorb excitation light incident thereon.

In some embodiments, the aperture is a window formed from a material that is transparent to both excitation light and emission light, and the window is coated on one or both of its surfaces with coating that is antireflective for both the excitation light and the emission light incident thereon.

In some embodiments, the sample chamber comprises an assay pad disposed adjacent to the aperture of the sample chamber base and configured to hold the sample. In some cases, the assay pad can include a sponge. Here, an internal structure of the sponge is configured to forward scattered the excitation light received thereon from the light source. For instance, the sponge can include light-absorbing backing configured to absorb the forward scattered excitation light incident thereon.

In some embodiments, the fluorometer can include relay optics between the light source and the assay pad, and configured to image the light source onto the assay pad.

In all the above embodiments of the ingestible device, the fluorometer can include an excitation filter between the light source and aperture, the excitation filter configured to transmit a portion of the excitation light. In all the above embodiments of the ingestible device, the light source and the photodetector can be configured symmetrically with respect to an optical axis of the aperture in a plane that includes the optical axis, the light source's location and the photodetector's location.

In some embodiments, a portion of the housing can be inside the fluorometer between the light source and the photodetector. Here, the ingestible device can include a separator inside the fluorometer and extending between a first end of the separator adjacent to the portion of the housing disposed between the light source and the photodetector and a second end of the separator adjacent to the aperture of the sample-chamber base. A first combination of (i) a first side of the separator, (ii) a first portion of the housing that supports the excitation filter, and (iii) a first portion of the sample-chamber base defines a first enclosure of the fluorometer. A second combination of (i) a second side of the separator opposing the first side of the separator, (ii) a second portion of the housing that supports the emission filter, and (iii) a second portion of the sample-chamber base defines a second enclosure of the fluorometer. Additionally, the first enclosure is configured to impede excitation light scattered therein from reaching the photodetector without first interacting with the sample and then propagating through the second enclosure. In some cases, the separator can include a plate. In some cases, the housing can include the separator. In some cases, the first and second sides of the separator can include the same or another light-absorbing material as the one in the light-absorbing material of the inner surfaces of the housing configured to absorb excitation light incident thereon.

In the above embodiment of the fluorometer, a first aperture adjacent to the excitation filter defines an excitation path along which excitation light propagates through the first enclosure from the excitation filter to the aperture of the sample-chamber base without scattering off the housing, the sample-chamber base, or the first side of the separator. Here, a second aperture adjacent to the emission filter defines an emission path along which emission light propagates through the second enclosure from the aperture of the sample-chamber base to the excitation filter without scattering off the housing, the sample-chamber base, or the second side of the separator. In some cases, the housing can include a shelf that has two openings corresponding to the first and second apertures. The shelf is configured to support the excitation filter adjacent to the first aperture and the emission filter adjacent to the second aperture. In some cases, the fluorometer can include a first plate disposed adjacent to the excitation filter and having an opening corresponding to the first aperture, and a second plate disposed adjacent to the emission filter and having an opening corresponding to the second aperture. In addition, a pair of apertures adjacent to the aperture of the sample-chamber base is configured to reinforce the definition of the corresponding excitation path and emission path, where the pair of apertures has two openings that are disposed on separate sides of the separator. For example, the openings of the pair of apertures can be circular. As another example, the openings of the pair of apertures can be shaped as segments of a circle.

In some embodiments, the first portion of the sample-chamber base can have a smaller lateral extent from the separator than the aperture of the sample-chamber base, such that the first enclosure is shaped as a first tunnel path. Additionally, the second portion of the sample-chamber base has a smaller lateral extent from the separator than the aperture of the sample-chamber base, such that the second enclosure is shaped as a second tunnel path. In some cases, the ingestible device can include a first sequence of baffles which protrude into the first tunnel path from (i) a first series of locations of the first side of the separator, distributed from its first end adjacent to the excitation filter to its second end adjacent to the aperture of the sample-chamber base, and (ii) corresponding locations of the housing. The first sequence of baffles defines a first sequence of respective apertures that collectively define an excitation light path between the light source and the aperture of the sample-chamber base. Here, the ingestible device also includes a second sequence of baffles which protrude into the second tunnel path from (i) a second series of locations of the second side of the separator, distributed from its second end adjacent to the aperture of the sample-chamber base to the emission filter, and (ii) corresponding locations of the housing. The second sequence of baffles defines a second sequence of respective apertures that collectively define an emission light path between the aperture of the sample-chamber base and the photodetector. Moreover, the first and second sequences of baffles can include the same or another light-absorbing material as the one in the light-absorbing material of the inner surfaces of the housing and the first and second sides of the separator configured to absorb excitation light incident thereon.

In some embodiments, the light source can be in a first plane that includes an optical axis of the aperture; the photodetector can be in a second plane that includes the optical axis, the second plane being different from the first plane; the aperture can be a window formed from a material that is transparent to both excitation light and emission light; and the fluorometer can include a beam dump in the first plane and configured to absorb excitation light specularly reflected from the window.

In general, another innovative aspect of the subject matter described in this specification can be embodied in an ingestible device that includes a sample chamber configured to hold a sample, the sample chamber including a base having an aperture; and a fluorometer separated from the sample chamber by the sample chamber base, the fluorometer comprising (I) a light source configured to emit excitation light, (II) an excitation filter between the light source and aperture, the excitation filter configured to transmit a portion of the excitation light, (III) a first light guide configured to guide excitation light transmitted through the excitation filter to the sample, (IV) a photodetector configured to detect emission light generated via the interaction of the excitation light with the sample, (V) an emission filter between the aperture and the photodetector, the emission filter configured to (i) transmit the emission light incident thereon, and (ii) block excitation light incident thereon, and (VI) a second light guide configured receive the excitation light through the aperture and to guide the received excitation light to the emission filter. The ingestible device further includes a housing that houses the sample chamber and the fluorometer.

The foregoing and other embodiments of the ingestible device can each optionally include one or more of the following features, alone or in combination. In particular, one embodiment includes all the following features in combination.

In some embodiments, each of the first and second light guide can include one or more optical fibers. In some cases, either the first light guide or the second light guide or both includes an optical-fiber bundle.

In some embodiments, the sample chamber can include an assay pad disposed adjacent to the aperture of the sample chamber base and configured to hold the sample. In some cases, the first light guide can have an input end coupled with the excitation filter and an output end coupled with the assay pad. The first light guide is configured to (i) receive the excitation light at the input end, (ii) guide the excitation light from the input end to the output end, and (iii) deliver the excitation light through the output end to the assay pad. Additionally, the second light guide can have an input end coupled with the assay pad and an output end coupled with the emission filter. The second light guide is configured to (i) receive the emission light at the input end, (ii) guide the emission light from the input end to the output end, and (iii) deliver the emission light through the output end to the emission filter.

In some embodiments, the light source can be disposed on the same inner surface of the housing as the photodetector. In some embodiments, inner surfaces of the housing that are part of the fluorometer can include a light-absorbing material configured to absorb excitation light incident thereon.

In all the above embodiments of any of the foregoing ingestible devices, the fluorometer can have a longitudinal size in a range of 6-10 mm and a transverse size in a range of 8-13 mm. In all the above embodiments of any of the foregoing ingestible devices, the ingestible device can have a longitudinal size in a range of 20-30 mm and a transverse size in a range of 8-13 mm.

In all the above embodiments, any of the foregoing ingestible devices can include a sampling subsystem configured to retrieve the sample from the gastrointestinal (GI) tract of a subject in vivo.

In all the above embodiments of any of the foregoing ingestible devices, the light source can include one or more light emitting diodes.

In all the above embodiments of any of the foregoing ingestible devices, the photodetector can include one or more photodiodes or photomultipliers.

In all the above embodiments, any of the foregoing ingestible devices can include a power subsystem housed by the housing and configured to provide power to the light source and the photodetector. In all the above embodiments, any of the foregoing ingestible devices can include an electronics subsystem housed by the housing and configured to synchronize activation of the light source and readout of the photodetector.

In all the above embodiments of any of the foregoing ingestible devices, the emission filter can be a long-wavelength pass filter having a lower-bound wavelength shorter than wavelengths of the emission light. In some cases, the excitation filter can be a short-wavelength pass filter having an upper-bound wavelength longer than wavelengths of the excitation light, and shorter than the lower-bound wavelength of the emission filter.

In all the above embodiments of any of the foregoing ingestible devices, the assay pad can include absorbent material. In some cases, the absorbent material can include a sponge.

In general, another innovative aspect of the subject matter described in this specification can be embodied in a system including any of the foregoing ingestible devices; and a hardware processor configured to produce information about the sample based on characteristics of the detected emission light.

The subject matter described herein can be implemented in particular embodiments so as to realize one or more of the following advantages. For example, in certain embodiments, the coating of the housing and sample-chamber base (also called assay bulkhead) of the disclosed fluorometers can cause 175× cumulative reduction in the amount of scattered excitation light that can reach the photodetector. As another example, in some embodiments, specular reflection from the window, which separates the sample chamber from the disclosed fluorometers, directed toward the photodetector would be approximately 4% of incident light flux, based on the geometry and materials employed, if the window were uncoated. However, in such embodiments, the specular reflection from the window directed toward the photodetector may be reduced to <0.5% of incident light flux by applying an anti-reflection coating to the window.

As yet another example, in certain embodiments, the specular reflection from the window directed toward the photodetector, which is approximately 4% of incident light flux based on the geometry and materials employed, could be redirected away from the photodetector and into a light trap reducing the amount of excitation light reaching the photodetector as background light.

As yet another example, substantially all the emitted excitation light can be delivered to the sample by using some version of a light guide extending between the fluorometer and the sample chamber. As a further example, substantially all the unused excitation light can be blocked, and substantially all the emitted emission light can be collected by including inside the fluorometer a fluorescence-collection optic coupled with the sample chamber-based assay pad.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an ingestible device including a fluorometer.

FIGS. 2A-2S show aspects of an ingestible device including an example of a fluorometer.

FIGS. 3A-3B show aspects of an ingestible device including another example of a fluorometer.

FIGS. 4A-4C show aspects of an ingestible device including yet another example of a fluorometer.

FIGS. 5A-5B show aspects of an ingestible device including yet another example of a fluorometer.

FIG. 6 is a plot of normalized spectra corresponding to particular types of components used to fabricate the disclosed fluorometers.

Like reference numbers and designations in the various drawings indicate like elements.

### DETAILED DESCRIPTION

General features of ingestible devices and their use are described, for example, in the following U.S. patent applications, each of which is hereby incorporated by reference: U.S. Ser. No. 14/460,893, entitled “Ingestible Medical Device,” and filed Aug. 15, 2014; U.S. Ser. No. 15/514,413, entitled “Electromechanical Pill Device with Localization Capabilities,” and filed Mar. 24, 2017; U.S. Ser. No. 15/680,400, entitled “Systems and Methods for Obtaining Samples using Ingestible Devices,” filed on Aug. 18, 2017; U.S. Ser. No. 15/680,430, entitled “Sampling Systems and Related Materials and Methods,” filed on Aug. 18, 2017; U.S. Ser. No. 15/699,848, entitled “Electromechanical Ingestible Delivery of a Dispensable Substance,” filed on Sep. 8, 2017; U.S. Ser. No. 15/835,270, entitled “Gastrointestinal Tract Detection Methods, Device and Systems,” and filed Dec. 7, 2017; U.S. Ser. No. 15/835,237, entitled “Gastrointestinal Tract Detection Methods, Device and Systems,” and filed Dec. 7, 2017; U.S. Ser. No. 15/835,292, entitled “Gastrointestinal Tract Detection Methods, Device and Systems,” and filed Dec. 7, 2017; U.S. Ser. No. 15/844,349, entitled “Ingestible Device and Associated Methods,” filed Dec. 15, 2017; U.S. Ser. No. 15/844,381, entitled “Ingestible Device and Associated Methods,” filed Dec. 15, 2017; U.S. Ser. No. 15/844,427, entitled “Ingestible Device and Associated Methods,” filed Dec. 15, 2017; Ser. No. 15/694,458, entitled “Systems and Methods for Extracting a Sample from an Ingestible Device,” filed on Mar. 15, 2018; U.S. Ser. No. 15/940,407, entitled “Localization Systems and Methods for an Optoelectromechanical Pill Device,” filed on Mar. 29, 2018; U.S. Ser. No. 16/299,537, entitled “Ingestible Device With Relatively Large Payload Volume,” filed Mar. 12, 2019; and PCT/US2019/034795, entitled “Devices and Systems for Gastrointestinal Microbiome Detection and Manipulation,” and filed May 31, 2019.

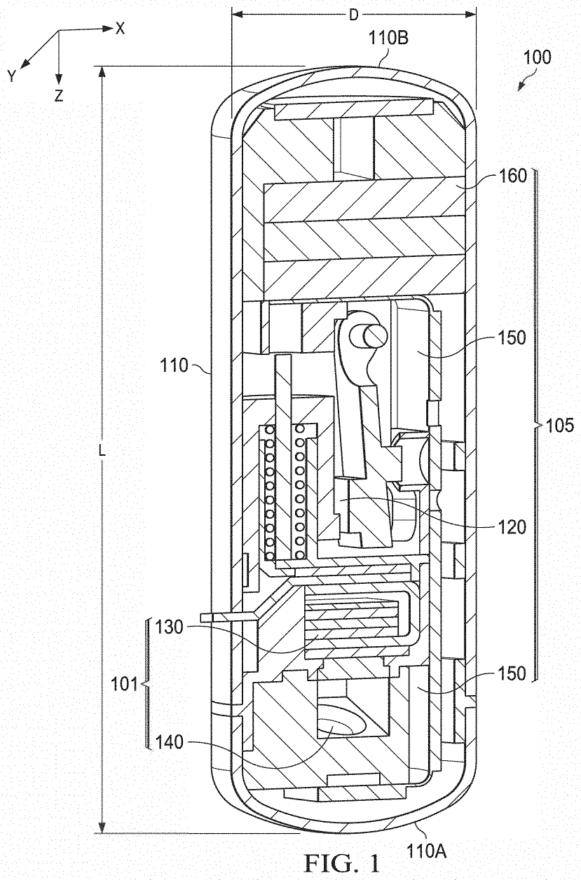

FIG. 1 is a cutaway diagram of an ingestible device 100 that includes a fluorometer 140. The fluorometer 140 and a sample chamber 130 are part of an assay system 101 of the ingestible device 100. Additionally, the ingestible device 100 includes a service system 105 that can have the following different subsystems: a power subsystem 160, an electronics and localization subsystem 150, and a sampling subsystem 120.

The ingestible device 100 may generally be in the shape of a capsule, like a conventional pill (e.g., like the example illustrated in FIG. 1), or alternatively in a different shape, such as, for example, the shape of a sphere. In general, the shape of the ingestible device is selected to provide for easier ingestion, and also to be familiar to healthcare practitioners and patients. In the example illustrated in FIG. 1, the ingestible device 100 has an outer housing with a first end 110A, a second end 110B, and a wall 110 extending longitudinally from the first end to the second end, e.g., along the z-axis. In some embodiments, the ingestible device is sized according to relevant standards, such as discussed, for example, in www.fda.gov/downloads/drugs/guidances/ucm377938.pdf and/or en.wikipedia.org/wiki/Capsule (pharmacy). In some embodiments, the ingestible device 100 can have a length L=25-30 mm, between the first and second ends 110A, 110B, and/or a diameter D=8-13 mm in a transverse plane, e.g., in the (x,y)-plane. Generally, at least the outer housing of the ingestible device 100 may be manufactured from a type of plastic, such as an acrylic polymer material. The outer housing, in effect, protects the interior of the ingestible device 100 from its external environment and also protects the external environment (e.g., the GI tract) from components inside the ingestible device.

The power subsystem 160 can include one or more batteries configured to power the electronics subsystem 150. The electronics subsystem 150 includes a microcontroller, optionally in signal communication with an external base station (not shown in FIG. 1). Examples of such arrangements are disclosed in one or more of the patent applications incorporated by reference herein. The electronics subsystem 150 can include one or more of a processing unit to process intensity and/or spectrally-selective intensity data produced by the fluorometer 140, memory to store the intensity and/or spectrally-selective intensity data, and one or more environmental sensors. Further, the power subsystem 160 is configured to power one or more light sources and photodetectors that are part of by the fluorometer 140. Furthermore, the electronics subsystem 150 includes electronic circuitry to synchronize activation of the one or more light sources and readout of the one or more photodetectors.

The sampling subsystem 120 is configured to take in a sample from the environment exterior to ingestible device 100, e.g., from the GI tract, and includes one or more ports, valves, pumps and/or conduits. For instance, the components of the sampling subsystem 120 provide fluid communication between the sample chamber 130 and the exterior of ingestible device. In operation, when the ingestible device 100 determines the device itself arrives at a target location within the GI tract, the sampling subsystem 120 takes in a sample from the target location. For example, fluid from the GI tract can enter the device 100 via a port into the sample chamber 130. Exemplary sampling systems are disclosed, for example, in one or more of the co-pending U.S. patent applications incorporated by reference herein. Exemplary localization systems as disclosed, for example, in one or more of the co-pending U.S. patent applications incorporated by reference herein.

Intensity data or spectrally-selective intensity data or both for light emitted by a sample in the sample chamber 130 can be produced in-situ by the on-board fluorometer 140, in the following manner. The fluorometer 140 excites the sample with excitation light and collects emission light emitted by the excited sample. The emission light from the excited sample is collected by the fluorometer 140 to yield intensity data and spectrally-selective intensity data. The data can be stored and/or processed in-situ by the electronics subsystem 150 of the ingestible device 100, and/or can be transmitted to a receiver remote from the ingestible device. Various embodiments of fluorometers described below can be incorporated in the ingestible device 100. As described in more detail below, components of the ingestible device 100 can be designed so that the intensity data and/or spectrally-selective intensity data is produced with sufficient sensitivity to characterize any of a variety of GI tract samples.

### Example 1

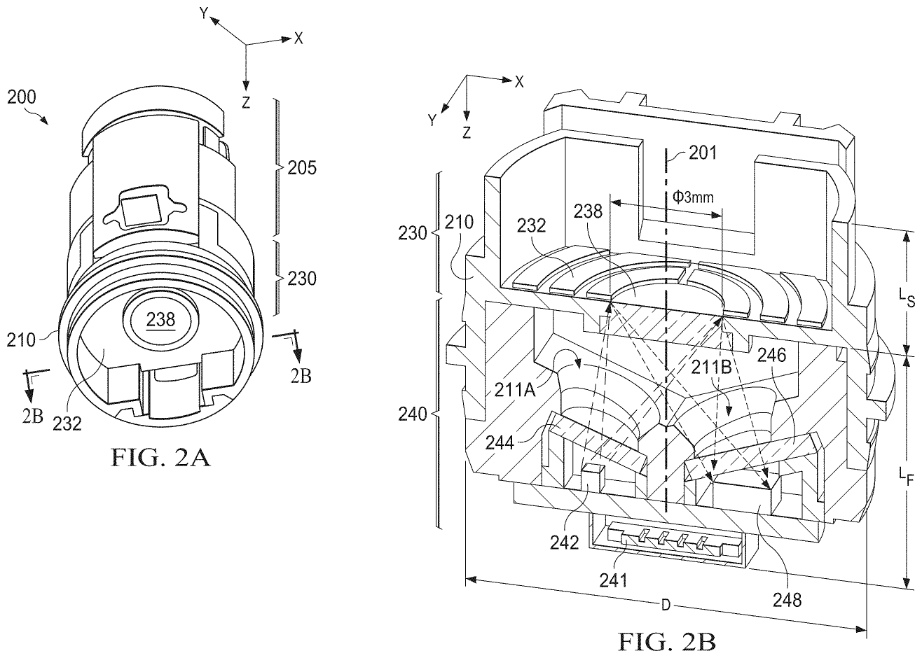

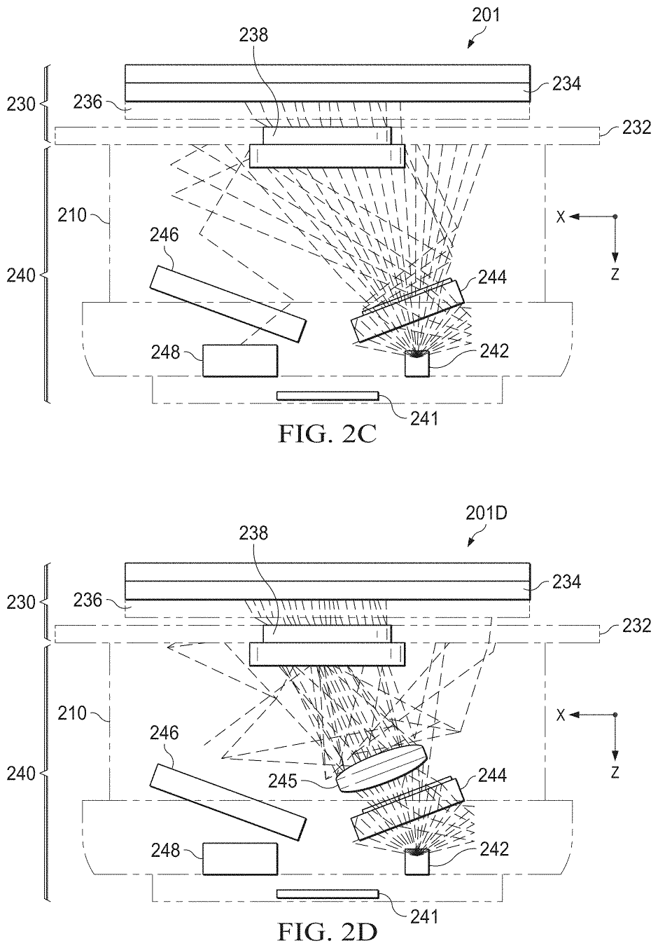

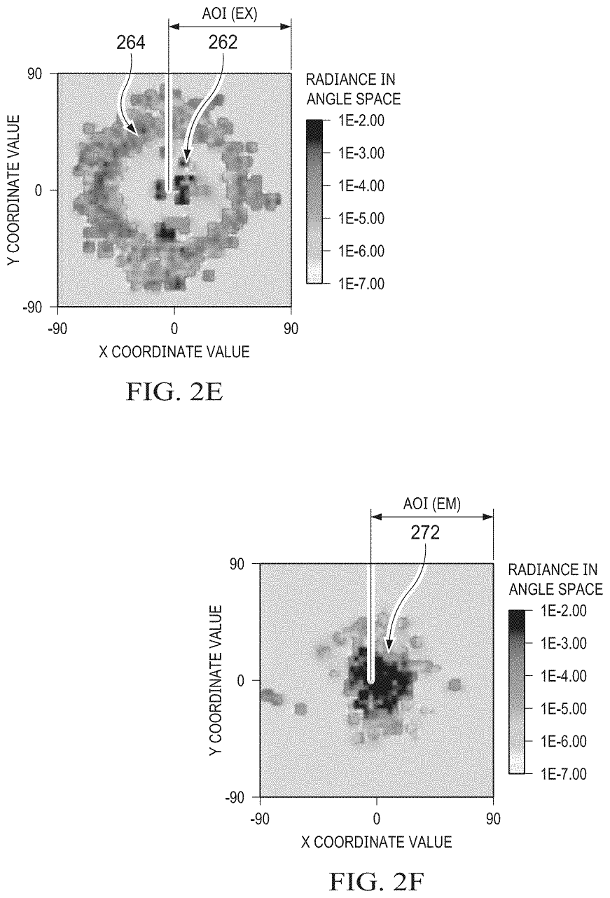

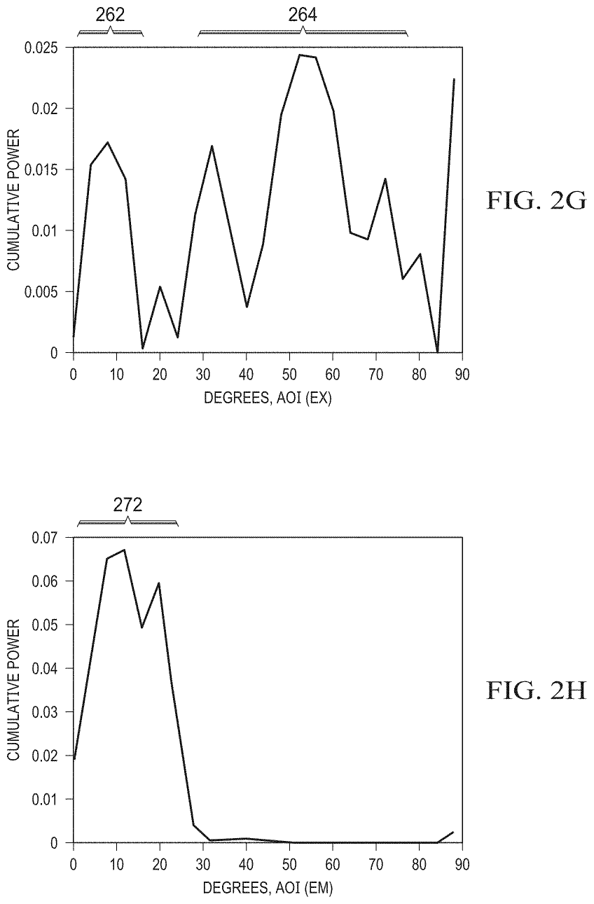

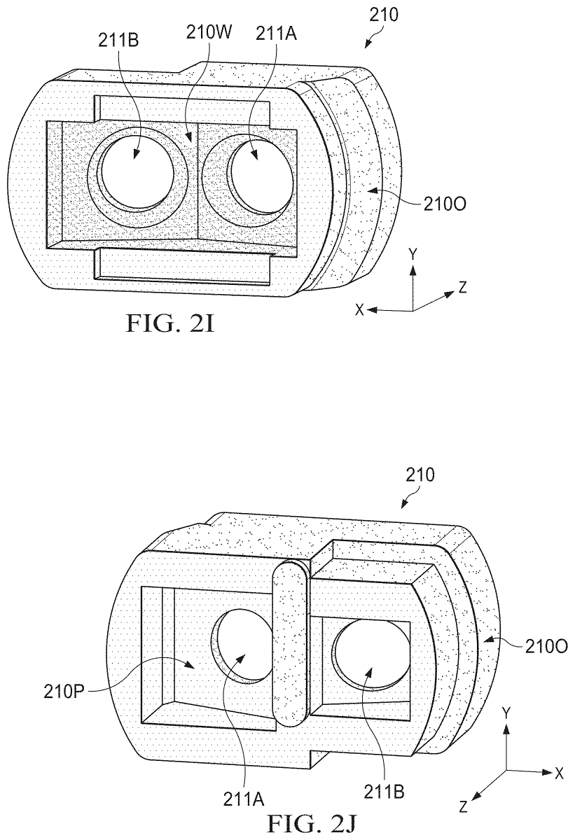

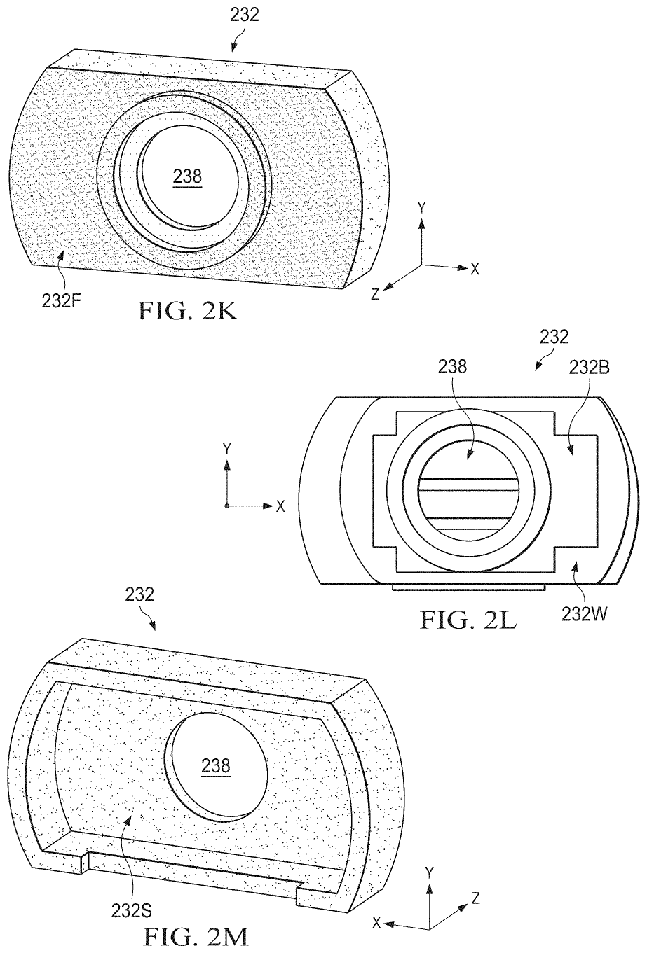

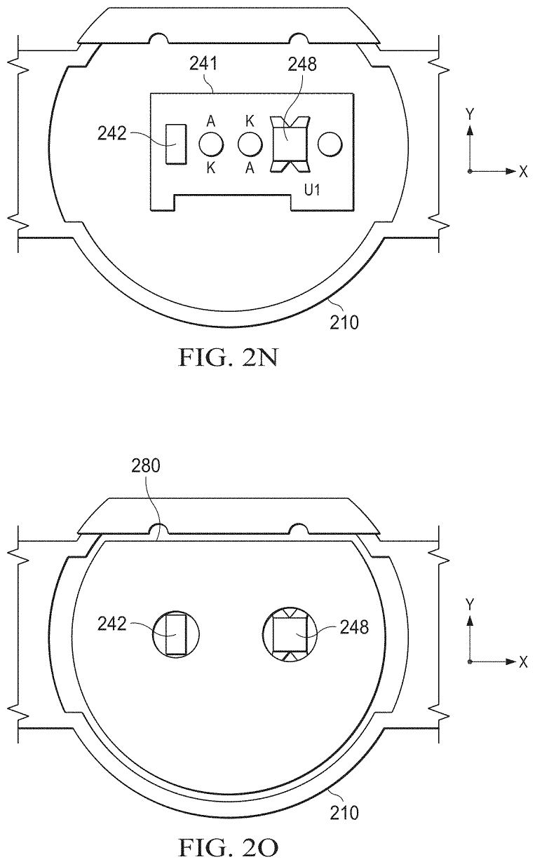

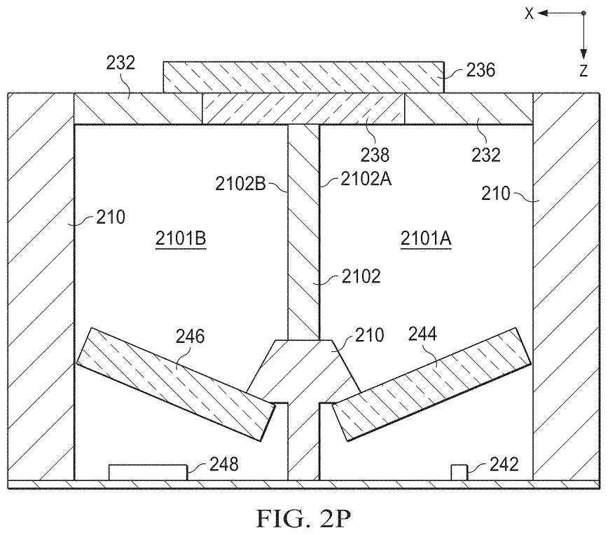

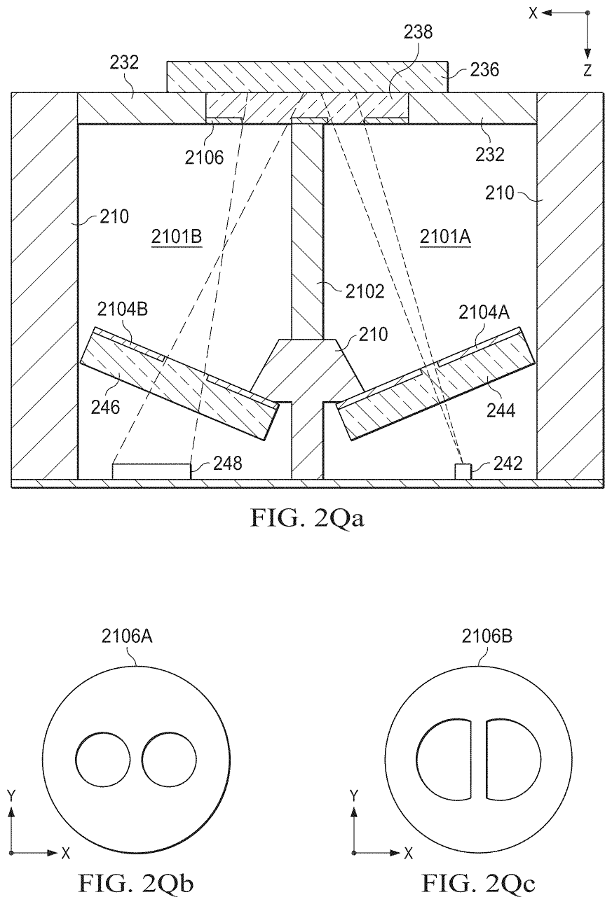

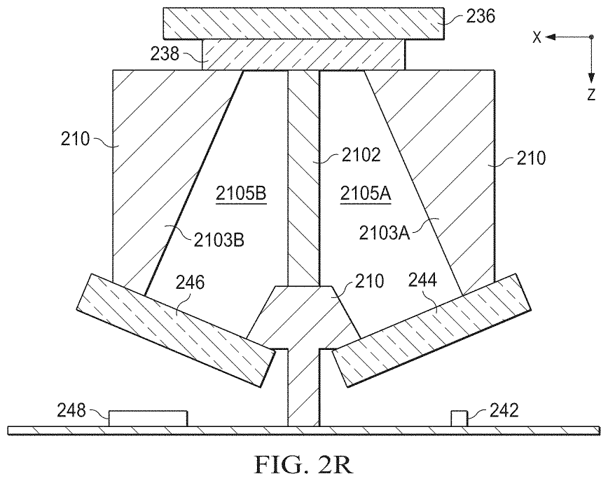

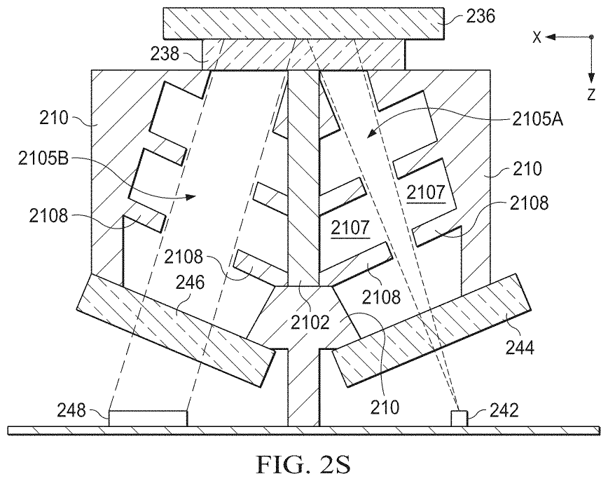

FIGS. 2A and 2B are cutaway diagrams showing different portions of an ingestible device 200 that includes a fluorometer 240. In addition to the fluorometer 240, the ingestible device 200 also includes a sample chamber 230 and a service system 205. The service system 205 can be implemented as the service system 105 described above in connection with FIG. 1. Housing 210 encloses the service system 205, the sample chamber 230 and the fluorometer 240 of the ingestible device 200. FIG. 2C is a cutaway diagram of an assay system 201 of the ingestible device 200. In FIG. 2B, a perspective view of the assay system 201 is shown. The assay system 201 includes the sample chamber 230 and fluorometer 240.

Referring now to FIGS. 2A-2C, the sample chamber 230 is separated from the fluorometer 240 by a sample-chamber base 232, here disposed in the (x,y)-plane, which is designed to allow both emission light and excitation light to pass between the fluorometer 240 and the sample chamber 230. The sample-chamber base 232 can be formed from, for example, plastic or aluminum or a combination of these materials. The sample-chamber base 232 has an aperture covered by an assay window 238. In general, the diameter (clear aperture) of the assay window 238 can be as large as the inner diameter of the outer housing 100 or as small as is appropriate based on (i) constraints relating to signal light to background light ratio and (ii) constraints relating to assay photobleaching. In some embodiments, the assay window 238 can have a diameter of 1-5 mm, e.g., 3 mm, and is made from a material that is transparent to both excitation light and emission light. For example, the assay window 238 can be fabricated using injection molding from a clear polycarbonate of appropriate grade. As another example, the assay window 238 can be fabricated using 3D-printing from a material known as SOMOS® available from WaterShed.

In some implementations, a sample—retrieved in the sample chamber 230 from the GI tract—is held by an assay pad 236 disposed adjacent to the assay window 238. In turn, the assay pad 236 is sandwiched by the assay window 238 on one side and a wicking pad 234 on the other side. The assay pad 236 can have a thickness of 0.2-0.6 mm and a diameter of 4-8 mm. In some implementations, the assay pad 236 can be made from an absorptive material, e.g., a sponge. In general, the sample is at least partially absorbed in the absorptive material of the assay pad 236.

In general, the sample chamber 230 and the fluorometer 240 may be sized as appropriate. In some embodiments, a size of each of the sample chamber 230 and the fluorometer 240 in the transverse plane of the ingestible device 200, e.g., the (x,y)-plane, can be D=8-13 mm. In some embodiments, a size of the sample chamber 230 along the longitudinal direction can be L_{S}=1-3 mm. In some embodiments, a size of the fluorometer 240 along the longitudinal direction of the ingestible device 200, e.g., along the z-axis, is L_{F}=6-10 mm. In certain embodiments, the sample chamber extends along the entire length of the capsule.

The fluorometer 240 includes a light source 242 configured to emit excitation light, and a photodetector 248 configured to detect emission light. The light source 242 can be implemented as a light emitting diode (LED), a tunable LED or laser, e.g., as a QT BrightTek LED. In some implementations, the light source produces light at one or more wavelengths in the Ultraviolet, Visible, Near-infrared and/or Mid-infrared. In some implementations, the light source produces a continuous output and/or a pulsed output. The photodetector 248 can be implemented as one or more photodiodes, or photomultipliers (PM), e.g., as a SensL MicroFC-10010-SMT-TA (SiPM®). For example, the size in the (x,y)-plane of a (SiPM®) can be 1 mm×1 mm. The photodetector 248 may be made out of materials known in the art such as, but not limited to, silicon, germanium, indium gallium arsenide, lead (II) sulfide and/or mercury cadmium telluride. In some implementations, the photodetector detects light at one or more wavelengths in the Ultraviolet, Visible, Near-infrared and/or Mid-infrared.

The light source 242 and photodetector 248 are adjacent to a printed circuit board (PCB) 241, and are electrically coupled to respective electronic circuitry printed on the PCB 241. In the example shown in FIGS. 2A-2C, the light source 242 and photodetector 248 are disposed side-by-side in a transverse plane of the ingestible device 200, e.g., parallel to the (x,y)-plane. The PCB 241 is designed to include features configured to reduce or substantially eliminate reflection of light impinging inadvertently on the PCB 241 outside areas of the light source 242 and photodetector 248. In some implementations, a material from which the PCB 241 is fabricated can be Black FR-4. In some implementations, the PCB 241 can include black solder mask. In some implementations, the PCB 241 includes buried traces. In some implementations, at least some vias can be placed on “non-optical regions” of the PCB 241, e.g., away from the light source 242 and/or photodetector 248. If some vias are to be placed on “optical active regions” of the PCB 241, e.g., adjacent to the light source 242 and/or photodetector 248, then these vias can be filled. In some implementations, the PCB 241 can include thick copper ground planes to serve as an internal baffle. For example, in certain embodiments, such thick copper ground planes can have one ounce of copper per square foot of area, which corresponds to a thickness of approximately 35 microns. In other implementations, e.g., when such thick copper ground planes would interfere with a loop antenna of the ingestible device 100, the PCB 241 the ground planes could be configured as interleaved half-toned metallization on adjacent layers.

The fluorometer 240 also includes an excitation filter 244 disposed between the light source 242 and the assay window 238, and an emission filter 246 disposed between the assay window and the photodetector 248.

The light source 242 is configured to emit excitation light having a spectrum that overlaps an absorption spectrum of the sample held by the assay pad 236. The light source 242 is configured to emit the excitation light along a prevalent direction extending from the light source to the assay window 238 through a first aperture 211A of an internal surface of the housing 210. Typically, the excitation filter 244 is a short-wavelength-pass (SWP) filter or a bandpass filter. For example, the filter 244 is configured to pass light having wavelengths shorter than an upper-bound wavelength λ_{S} and to block light having wavelengths longer than that. Here, an excitation spectrum of the excitation light extends over wavelengths shorter than the upper-bound wavelength λ_{S}. The excitation light provided to the sample in this manner excites the sample, so the sample emits emission light through the assay window 238 back into the fluorometer 240. A portion of the emission light is emitted towards the photodetector 248 along a prevalent direction extending through a second aperture 211B of the internal surface of the housing 210, and then through the emission filter 246. The emission filter 246 is a bandpass filter (although a long-wavelength-pass (LWP) filter can be used) configured to pass light having wavelengths longer than a lower-bound bound wavelength λ_{L} and to block light having wavelengths shorter than that. Here, an emission spectrum of the emission light extends over wavelengths longer than the lower-bound wavelength λ_{L}.

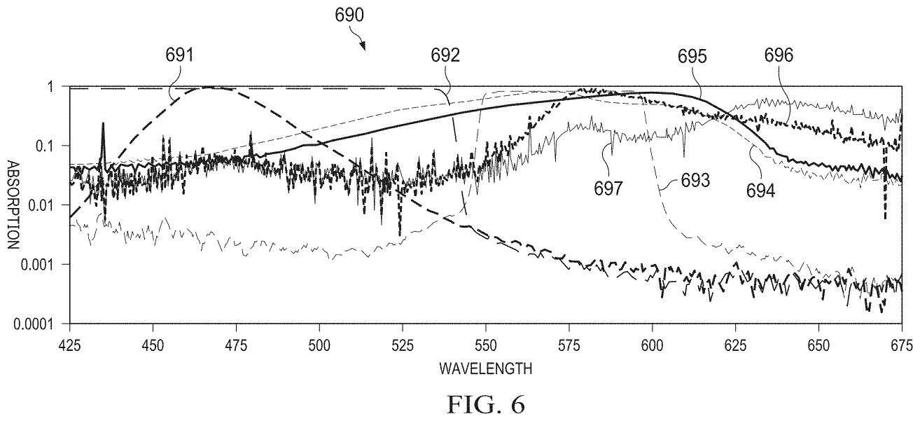

Various implementations of the fluorometer 240 can be fabricated using multiple types of the light source 242, the excitation filter 244, and the emission filter 246. Examples of these types of components are discussed below in connection with FIG. 6, which is a plot 690 of normalized spectra corresponding to a particular type of the light source 242, a particular type of the excitation filter 244, and a particular type of emission filter 246 used to fabricate the fluorometer 240. In some implementations, the light source 242 of the fluorometer 240 can be a light-emitting diode (LED), VAOL-S4SB4 LED SMD 0402, which emits blue light. Normalized spectrum 691 corresponds to light emitted by the VAOL-S4SB4 LED SMD 0402 light source 242 driven with a 10 mA drive current. This type of light source 242 has superior performance, compared to other types of blue LEDs, in terms of the signal light to background light ratio associated with the fluorometer 240. In some implementations, the excitation filter 244 of the fluorometer 240 can be a SWP filter specified by StarFish Medical and designed and fabricated by Evaporated Coatings Inc. (ECI). Normalized spectrum 692 corresponds to light transmitted through the ECI custom filter. This type of excitation filter 244 has a 50% transmission-level cutoff at 542.5 nm, and has an average transmission of 0.062% over 550-800 nm. In some implementations, the emission filter 246 of the fluorometer 240 can be a bandpass filter formed from a LWP filter with a cutoff at 550 nm, and a SWP filter with a cutoff at 600 nm. Normalized spectrum 693 corresponds to light transmitted through this bandpass filter centered at 575 nm with a bandpass width of 50 nm. This type of emission filter 246, when fabricated on a low auto-fluorescence substrate, has superior performance, compared to other types of bandpass filters, in terms of the signal light to background light ratio associated with the fluorometer 240.

In addition to the noted spectra 691, 692, and 693, the graph 690 includes the following other spectra. Normalized spectrum 694 corresponds to light transmitted through a 10 mm path-length of Resorufin 0.03 nM:PBS, and normalized spectrum 695 corresponds to light transmitted through a 10 mm path-length of Resazurin 0.03 nM:PBS. Normalized spectrum 696 corresponds to light emitted by Resorufin 0.03 nM:PBS, and spectrum 697 corresponds to light emitted by Resazurin 0.03 nM:PBS normalized relative to the peak of spectrum 696. The disclosed fluorometer 240 of the ingestible device 200 can be configured to detect an amount of Resorufin in order to determine a corresponding amount of bacteria absorbed by the assay pad 236 from the GI tract of the person who swallows the ingestible device 200. Referring to FIG. 6, the spectrum 691 of the light source 242 and the spectra 692, 693 of the filters are chosen based on the spectra 694, 696 of the product dye and the spectra 695, 697 of the parent dye.

Additionally, note in the graph 690 that the spectrum 691 has a small but finite (non-zero) contribution in the bandpass of the spectrum 693. This small contribution corresponds to a portion of the light emitted by the light source 242 that would be transmitted by the emission filter 246 if this portion of the light emitted by the light source 242 were able to propagate to the emission filter 246. However, note in the graph 690 that the small contribution of the spectrum 691 over the bandpass of the spectrum 693 overlaps the long-wavelength portion of the spectrum 692. This means that the portion of light emitted by the light source 242 over the spectral range 550-600 nm is blocked by the excitation filter 244 before this portion of the light emitted by the light source 242 could reach the assay window 238 and be specularly reflected toward the photodetector 248.

The excitation filter 244 can have a thickness of less than 1 mm, e.g., 0.55 mm, and be made from, for example, glass with low auto-fluorescence. The excitation filter 244 is an interference filter. Therefore, its upper-bound wavelength λ_{S} blue-shifts for excitation light with increasing angles of arrival off-normal. This may lead to lower throughput of excitation light to the assay pad 236 at longer wavelengths of the excitation light. The emission filter 246 can have a thickness of less than 1 mm, e.g., 0.55 mm, and be made from glass with low auto-fluorescence. The emission filter 246 is an interference filter. Therefore, its lower-bound wavelength λ_{L} blue-shifts for excitation light with increasing angles of arrival off-normal. This may lead to leakage of stray excitation light to the photodetector 248, which is an effect that interplays detrimentally with non-collimated excitation light and reflective housing materials. Additionally, both the excitation filter 244 and the emission filter 246 of the fluorometer 240 have their edges blacked to further reduce stray light.

During operation, the emission filter 246 blocks excitation light that reaches the emission filter after spurious scattering off the internal surface of the housing 210 of the fluorometer 240, and transmits emission light that reaches the emission filter from the sample, after propagating through the assay window 238 and the free space between the assay window and the emission filter. Emission light collected by the photodetector 248 after transmission through the emission filter 246 can be used to determine information about the sample held by the assay pad 236. For example, the intensity of the emission light collected by the photodetector 248 is indicative of a quantity of the sample. As another example, the spectrum of the emission light collected by the photodetector 248 is indicative of the identity of the sample.

Because the light source 242 emits the excitation light within a large angular range, e.g., in accordance with a Lambertian angular distribution, and further since the excitation light transmitted through the excitation filter 244 propagates toward the assay window 238 through free space, a fraction of the excitation light may miss the assay window. Some of the excitation light that misses the assay window 238 may instead reach the sample-chamber base 232 adjacent to the assay window. And from there, the excitation light can scatter throughout the fluorometer 240, e.g., towards the photodetector 248. To reduce the fraction of the excitation light that misses the assay window 238, the fluorometer 240 can be modified by adding relay optics to modify divergence of the light transmitted by the excitation filter 244. FIG. 2D shows an assay system 201D which includes the sample chamber 230 (described above) and a fluorometer 240D. The fluorometer 240D can include the same components as the fluorometer 240 (described above). In addition, the fluorometer 240D includes relay optics 245 disposed between the excitation filter 244 and the assay window 238. The relay optics 245 can be implemented as one or more lenses. In some implementations, the relay optics 245 are configured such that the light source 242 is located at a focal point of the relay optics. Here, the relay optics 245 receive the diverging excitation light after transmission through the excitation filter 244 and collimate the received excitation light, i.e., form an image of the light source 245 at infinity, through the assay window 238 and assay pad 236. In other implementations, the relay optics 245 are between the light source 242 and the assay window 238, such that they image the light source through the assay window on the assay pad 236. In either of these implementations of the fluorometer 240D, the fraction of excitation light that misses the assay window 238 is smaller than in the case of the fluorometer 240, which does not use relay optics between its excitation filter 244 and assay window 238. For instance, in some embodiments, when the relay optics 245 are placed in the excitation path to image the light source 242 to the assay pad 236, the amount of emission light that reaches the photodetector 248 increases by 2.5×, however the amount of scattered excitation light that can reach the photodetector also increases by 1.6×. In combination, these effects result in an overall increase in sensitivity of the emission light detection.

To further improve the photodetector 248's emission light detection, e.g., as quantified through the ratio of signal light to background light, various aspects of components of the fluorometer 240 can be modified to increase the signal light and decrease the background light. First it was determined, through simulations, that most of background light is due to (i) scattering of excitation light from various internal surfaces of the housing of the fluorometer 240, (ii) specularly reflecting of excitation light from the fluorometer-side surface of the sample-chamber base 232, and (iii) backscattering of excitation light from the assay pad 236. Some results of the simulations are shown in FIGS. 2E-2H.

FIG. 2E is a false-color map of excitation light radiance versus an angle of incidence of the excitation light at the surface of the emission filter 246 facing the assay window 238. The angular coordinate corresponding to the vertical axis is orthogonal to the angular coordinate corresponding to the horizontal axis. FIG. 2G is a plot of a cross-section AOI(EX) of the false-color map in FIG. 2E. The central portion 262 of the excitation light radiance corresponds to excitation light that reaches the emission filter 246 after being specularly reflected from the assay window 238 and/or backscattered from the assay pad 236. The outer ring portion 264 of the excitation light radiance corresponds to excitation light that reaches the emission filter 246 after being scattered from the inner surfaces of the fluorometer 240's housing. Excitation light incident on the emission filter 246 in accordance with the simulation results shown in FIGS. 2E and 2G will contribute to the background light for the emission light detection by the photodetector 248. As noted above, the emission filter 246's effectiveness in blocking the excitation light decreases as the incident angle of the excitation light increases. Hence, the excitation light scattered by the housing—corresponding to the outer ring portion 264 of the excitation light radiance—will have a stronger contribution to the background light than the excitation light specularly reflected by the assay window 238 and/or backscattered from the assay pad 236—corresponding to the central portion 262 of the excitation light radiance.

FIG. 2F is a false-color map of emission light radiance versus an angle of incidence of the emission light at the surface of the emission filter 246 facing the assay window 238. FIG. 2H is a plot of a cross-section AOI(EM) of the false-color map in FIG. 2F. The excitation light radiance corresponding to emission light that reaches the emission filter 246 after propagating from the assay window 238 only has a central portion 272. Emission light incident on the emission filter 246, in accordance with the simulation results shown in FIGS. 2F and 2H, will be transmitted by the emission filter and, thus, will contribute as signal light for the emission light detection by the photodetector 248.

The high incident angle portions (corresponding to the radiance portion 264 in FIGS. 2E and 2G) of the excitation light that reaches the emission filter 246 can be reduced by treating or coating the internal surface of the housing 210 and the sample-chamber base 232 to be as absorbing as possible to the excitation light.

In some implementations, the housing 210 can be shaped, e.g., through 3D printing or injection molding, using black dyed polycarbonate, e.g., Covestro Makrolon® 2458-901528. For instance, 0.55 mm of Covestro Makrolon® 2458-901528 polycarbonate has an optical density (OD) of 5.5. In some implementations, to maximize its surface area, the internal surface of the housing 210 can be finished as rough as possible, within applicable limits of an injection molding process. In some implementations, the internal surface of the housing 210 can be coated using a whole-part chemical immersion process, e.g., to cover the housing's internal surface with Epner Laser Black. In this manner, the reflectivity of the housing 210's internal surface is reduced from about 50% to about 1%. This causes a factor 80× reduction in the amount of scattered excitation light that can reach the photodetector 248, but is accompanied by about 20% reduction of the amount of emission light that reaches the photodetector for the embodiment shown. In some implementations, at least some of the internal surfaces of the housing 210 can be coated with Acktar Light Absorbent Foil. FIG. 2I is a perspective view of a window-facing internal surface 210W of the housing 210. Here, portions of the window-facing internal surface 210W of the housing 210 adjacent to the first and second apertures 211A, 211B, represented with cross-hashed pattern (blue), will be black coated in a manner fully compliant with the specifications. Remaining portions of the window-facing internal surface 210W of the housing 210 distal from the first and second apertures 211A, 211B, represented with slanted-hashed pattern (red), will have relaxed specifications with regards to blackness. FIG. 2J is a perspective view of a PCB-facing internal surface 210P of the housing 210. Most of the PCB-facing internal surface 210P of the housing 210, represented with slanted-hashed pattern (red), will have relaxed specifications with regards to blackness. The outer surfaces 2100 of the housing 210 and a portion of the PCB-facing internal surface 210P of the housing 210, represented in both FIGS. 2I-2J with dotted-hashed pattern (green), have no requirements relating to blackness. As such, these portions of the housing 210 can be coated, or partially coated, with black film, or not coated at all. In some implementations, the portions of the surface of the housing 210 on which no black coating is applied, e.g., represented with dotted-hashed pattern (green), will be roughened appropriately.

In some implementations, the sample-chamber base 232 can be shaped, e.g., through 3D printing or injection molding, using black dyed polycarbonate, e.g., Covestro Makrolon® 2458-901528. In some implementations, to maximize its surface area, the internal surface of the housing 210 can be finished as rough as possible, within applicable limits of an injection molding process. At least portions of the fluorometer-facing surface of the sample-chamber base 232 can be coated with Acktar Light Absorbent Foil. In this manner, the reflectivity of the coated portions of the sample-chamber base 232 is reduced from about 80% to about 1%. This causes a factor 2× reduction in the amount of scattered excitation light that can reach the photodetector 248, but is accompanied by some reduction of the amount of emission light that reaches the photodetector for the embodiment shown. In combination, the foregoing coating causes about 175× cumulative reduction in the amount of scattered excitation light that can reach the photodetector 248.

The low incident angle portions (corresponding to the radiance portion 262 in FIGS. 2E and 2G) of the excitation light that reaches the emission filter 246 can be reduced in a variety of ways. For example, the assay window 238 can be coated with anti-reflecting film on both sides, so the assay window's reflectivity is reduced from about 4% to substantially zero (e.g., to five decimal points). This could result in 20% increase in signal light because more excitation light can reach the assay pad 236, and more emission light can get out through the assay window 238. Additionally, this could result in 80% decrease in background light because less excitation light can specularly reflect off the front surface of the assay window 238 towards the photodetector 248.

FIG. 2K is a perspective view, and FIG. 2L is a plan view, of a fluorometer-facing surface 232F of the sample-chamber base 232. Either the entire fluorometer-facing surface 232F of the sample-chamber base 232, as shown in FIG. 2K, or only a region 232B adjacent the aperture 238 and circumscribed by the housing interior, will be black coated. The portions of the fluorometer-facing surface 232F of the sample-chamber base 232, represented with cross-hashed pattern (blue), will be black coated in a manner fully compliant with the specifications. The “walls” of the aperture 238, represented with slanted-hashed pattern (red), will have relaxed specifications with regards to blackness. FIG. 2M is a perspective view of a sample chamber-facing surface 232S of the sample-chamber base 232. The sample chamber-facing surface 232S of the sample-chamber base 232 and portions of the sample-chamber base 232 normal to the (x,y)-plane, represented with dotted-hashed pattern (green), have no requirements relating to blackness. As such, these portions of the sample-chamber base 232 can be coated, or partially coated, with black film, or not coated at all. In some implementations, the portions of the surface of the sample-chamber base 232 on which no black coating is applied, e.g., represented with dotted-hashed pattern (green), will be roughened appropriately.

As another example, to reduce the possibility of excitation light backscattering into the fluorometer 240 by the assay pad 236 (e.g., so that less than 0.5% of the incident excitation light is backscattered into the fluorometer 240 by the assay pad 236) the material of the assay pad can be configured to favor forward scattering of the excitation light. In addition, the assay pad can be provided with excitation light absorbing backing. In this manner, the background-light contribution of the assay pad backscattered emission light can drop to zero. However, a decrease by a factor 2× in the signal light can occur due to the fact that the excitation light will no longer be “sequestered” near the window side of the assay pad 238 to excite more of the sample held by the assay pad.

As yet another example, to reduce the possibility of excitation light propagating towards the photodetector 248, through multiple scattering events between the PCB 241 and either the PCB-facing internal surface 210P of the housing 210, and/or the light source-facing surface of the excitation filter 244—, (e.g., so that less than 0.5% of the incident excitation light undergoes such propagation toward the photodetector 248), a gasket can be placed between the PCB 241 and the filters 244, 246. FIG. 2N is a plan view of a portion of the housing 210 in which the PCB 241 is disposed. Here, the light source 242 and the photodetector 248 are supported by the PCB 241. FIG. 2O is a plan view of the same portion of the housing 210 in which a gasket 280 was disposed over the PCB 241. The gasket 280 has a first aperture aligned with the light source 242 to allow for emitted light to propagate from the light source 242 to the excitation filter 244, and a second aperture aligned with the photodetector 248 to allow for emission light to propagate from the emission filter 246 to the photodetector 248. In some implementations, the gasket 280 can be fabricated from closed cell foam material, e.g., Stockwell Elastomerics, 20A SE2020 with black pigment. The first and second apertures of the gasket 280 can be die cut to tight fit around the light source 242 and the photodetector 248. For instance, 0.8 mm of Stockwell Elastomerics, 20A SE2020 with black pigment has an optical density (OD) of 6.

Other ways to isolate the excitation light and prevent it from reaching the photodetector 248 include, but are not limited to, the following. They correspond to different configurations of the enclosure defined inside the fluorometer 240, see FIGS. 2B, 2I, and 2K, between the side walls of the housing 210, the window-facing internal surface 210W of the housing 210, and the fluorometer-facing surface 232F of the sample-chamber base 232.

FIG. 2P is a side view of a portion of an embodiment of the fluorometer 240 that includes, in addition to the components of the embodiment illustrated in FIG. 2B, a separator 2102. The separator 2102 is configured to impede, e.g., at least hinder, if not prevent altogether, the excitation light emitted by the light source 242 from propagating to the photodetector 248 without first interacting with the assay pad 236 adjacent to the first portion of the assay window 238, as explained below.

The separator 2102 extends, e.g., along the z-axis, from (i) a first end adjacent to a portion of the housing 210 disposed between, e.g., along the x-axis, the light source 242 and the photodetector 248, to (ii) a second end adjacent to the assay window 238 of the sample-chamber base 232. In this manner, the separator 2102 defines two enclosures inside the fluorometer 240.

A first enclosure 2101A is between (i) a first side 2102A of the separator 2102, (ii) a first portion of the housing 210 supporting the excitation filter 244 and extending from the first end of the separator 2102 to a first portion of the sample-chamber base 232, and (iii) the first portion of the sample-chamber base 232 supporting a first portion of the assay window 238 and extending from the second end of the separator 2102 to the first portion of the housing 210. A second enclosure 2101B is between (i) a second side 2102B of the separator 2102 opposing the first side 2102A, (ii) a second portion of the housing 210 supporting the emission filter 246 and extending from the first end of the separator 2102 to a second portion of the sample-chamber base 232, and (iii) the second portion of the sample-chamber base 232 supporting a second portion of the assay window 238 and extending from the second end of the separator 2102 to the second portion of the housing 210.

In some implementations, the separator 2102 is shaped as a plate, e.g., along the (y,z)-plane, to define a straight partition. A thickness of the separator 2102, e.g., a distance along the x-axis between the first and second sides 2102A, 2102B, can be in the range of 0.2-1.5 mm. In some implementations, the separator 2102 can be molded and, thus, be part of the housing 210.

The separator 2102 is opaque to the excitation light propagating to the first enclosure 2101A from the light source 242 through the excitation filter 244. In some implementations, the separator 2102 is also opaque to the emission light propagating to the second enclosure 2101B from the assay pad 236 through the assay window 238. For example, the separator 2102 can be fabricated from a darkened or black plastic to cause absorption of excitation light, and optionally of the emission light. An example of such material is black dyed polycarbonate, e.g., Covestro Makrolon® 2458-901528. As another example, the opposing sides 2102A, 2102B of the separator 2102 can be coated to further reduce reflection, for example using Epner Laser Black. The housing 210 and the sample-chamber base 232 are fabricated and coated, as described above, to reduce light reflection/scatter.

The excitation light can be further isolated from the photodetector 248 through the use of apertures as described next. FIG. 2Qa is a side view of a portion of another embodiment of the fluorometer 240 that includes, in addition to the components of the embodiment illustrated in FIG. 2P, multiple apertures 2104A, 2104B, and optionally 2106.

Here, a first aperture 2104A is disposed adjacent to the excitation filter 244 and is configured to further restrict the propagation direction of the excitation light by defining a first light cone, represented by short-dashed lines, between the light source 242 and the assay pad 236. Note that the term “aperture” denotes a structure having an opening from one side to the opposing side of the structure. Examples of aperture structures are provided below. A second aperture 2104B is disposed adjacent to the emission filter 246 and is configured to further restrict the propagation direction of the emission light by defining a second light cone, represented by long-dashed lines, between the assay pad 236 and the photodetector 248. Although the three-dimensional (3D) light paths, which are defined as illustrated in FIG. 2Qa, are referred to as light cones, other 3D shapes of the light paths, defined in this manner, are possible.

In some implementations, the apertures 2104A, 2104B can be part of the mechanical construction of the housing 210 as a molded part. Such an implementation was described above in connection with FIGS. 2B and 2I-2J, where aperture 211A corresponds to aperture 2104A, and aperture 211B corresponds to aperture 2104B. With reference to FIGS. 2B and 2Qa, each aperture 211A/2104A, 211B/2104B is a shelf having a respective opening and supporting a respective filter 244, 246. In other implementations, apertures 2104A, 2104B can be thin metal plates that have respective openings and are blackened for light absorption. For example, the metal plate can be implemented as a dark foil, with a thickness of 0.01-0.1 mm, for instance.

A pair of apertures 2106 can be disposed adjacent to the assay window 238 and is configured to reinforce the definition of the first, excitation light cone extending through the first enclosure 2101A, and to reinforce the definition of the second, emission light cone extending through the second enclosure 2101B. The pair of apertures 2106 has two openings that are disposed, e.g., in the (x,y)-plane, on separate sides of the separator 2102 to separate the excitation light cone from the emission light cone all the way down to the assay pad 236. Such separation of the excitation cone from the emission cone ensures that the photodetector 248 receives only light that is scattered through the assay pad 236.

FIG. 2Qb is a plan-view of an embodiment 2106A of the pair of apertures to be disposed adjacent to the assay window 238, e.g., in the (x,y)-plane. Here, the two openings corresponding to the first and second enclosures 2101A, 2101B, have circular shapes. Other shapes of the two openings of the pair of apertures are possible. FIG. 2Qc is a plan-view of another embodiment 2106B of the pair of apertures to be disposed adjacent to the assay window 238, e.g., in the (x,y)-plane. Here, the two openings corresponding to the first and second enclosures 2101A, 2101B, are shaped as segments of a circle, each with a wider area than the area of corresponding openings of the embodiment 2106A of the pair of apertures. The embodiment 2106B of the pair of apertures separates the excitation cone from the emission cone, but also allows more total excitation light to reach the assay pad 236 through the excitation cone, and more total emission light to reach the photodetector 248 through the emission cone. Each of the embodiments 2106A, 2106B of the pair of apertures can be openings in a thin metal plate, blackened for light absorption. For example, the metal plate can be implemented as a dark foil, with a thickness of, e.g., 0.01-0.1 mm.

Notwithstanding the foregoing, the presence of the pair of apertures 2106 is not dependent on having the separated-chamber embodiment with the separator 2102. For instance, in other implementations, the pair of apertures 2106, or a differently shaped aperture, can be disposed adjacent to the assay window 238 into an open chamber, such as the one illustrated in FIG. 2B.

Referring again to FIG. 2P, the excitation light can be further isolated from the photodetector 248 by narrowing the first and second enclosures 2101A, 2101B to tunnel paths, as described next. FIG. 2R is a side view of a portion of yet another embodiment of the fluorometer 240 that includes the components of the embodiment illustrated in FIG. 2P, and in which the first and second enclosures 2101A, 2101B have been narrowed to corresponding first and second tunnel paths 2105A, 2105B. Here, the housing 210 is extended perpendicular to the separator 2102, e.g., along the x-axis, into the first and second enclosures 2101A, 2101B, and thus closer to the corresponding sides of the separator 2102.

In the example shown in FIG. 2R, the first tunnel path 2105A is between (i) the first side 2102A of the separator 2102, (ii) a first portion of the housing 210 supporting the excitation filter 244 and extending from the first end of the separator 2102 to a first portion of the assay window 238, and (iii) the first portion of the assay window 238 extending from the second end of the separator 2102 to the first portion of the housing 210. The second tunnel path 2105B is between (i) the second side 2102B of the separator 2102 opposing the first side 2102A, (ii) a second portion of the housing 210 supporting the emission filter 246 and extending from the first end of the separator 2102 to a second portion of the assay window 238, and (iii) the second portion of the assay window 238 extending from the second end of the separator 2102 to the second portion of the housing 210. The housing 210 and the separator 2102 are fabricated and coated, as described above, to reduce light reflection/scatter.

The excitation light can be further isolated from the photodetector 248 by using baffles, also referred to as fins, to narrow the first and second tunnel paths, as described next. FIG. 2S is a side view of a portion of yet another embodiment of the fluorometer 240 that includes the components of the embodiment illustrated in FIG. 2R, and baffles 2108 configured to suitably narrow the first and second tunnel paths 2105A, 2105B.

A first sequence of baffles 2108 protrude into the first tunnel path 2105A from (i) a first series of locations of the first side of the separator 2102, distributed from its first end adjacent to the excitation filter 252 to its second end adjacent to the assay window 238, and (ii) corresponding locations of the housing 210 to define a first sequence of respective apertures that collectively define an excitation light cone, represented by short-dashed lines, between the light source 242 and the assay pad 236. A second sequence of baffles 2108 protrude into the second tunnel path 2105B from (i) a second series of locations of the second side of the separator 2102, distributed from its second end adjacent to the assay window 238 to its second end adjacent to the emission filter 254, and (ii) corresponding locations of the housing 210 to define a second sequence of respective apertures that collectively define an emission light cone, represented by long-dashed lines, between the assay pad 236 and the photodetector 248. Note that the space between adjacent baffles of either the first or second sequences of baffles are referred to as open recesses 2107.

Here, excitation light, which does not pass through the baffles 2108 of the first sequence in accordance with the excitation light cone, is trapped in corresponding open recesses 2107. Multiple bounces (reflection or scatter) are needed to exit each of the recesses 2107 and get back excitation light into excitation light cone. In this manner, each bounce reduces the amount of scattered or stray excitation light. Similarly, emission light and stray/leakage excitation light which does not pass though baffles 2108 of the second sequence, in accordance with the emission light cone, is trapped in corresponding open recesses.

In some implementations, the baffles 2108 can be part of the mechanical construction of the separator 2102 and the housing 210 as molded parts. In other implementations, the baffles 2108 can be inserted in the separator 2102 and the housing 210 as plates, or similar structures, that are blackened for light absorption. For example, the metal plates can be implemented as dark foils, with a thickness of 0.01-0.1 mm, for instance. The housing 210 and corresponding baffles 2108 coupled thereto, and the separator 2102 and corresponding baffles 2108 coupled thereto are fabricated and coated, as described above, to reduce light reflection/scatter.

As yet another example of a way to reduce the amount of background light that reaches the photodetector 248, note that for the fluorometer 240, the light source 242 and the photodetector 248 are disposed (i) side-by-side in a transverse plane, e.g., parallel to the (x,y)-plane, along a diameter of the ingestible device 200, and (ii) symmetrically relative to an optical axis 201 of the assay window 238, e.g., parallel to the z-axis. This relative orientation of the light source 242, the assay window 238 and the photodetector 248 causes excitation light emitted by the light source to specularly reflect off the assay window directly towards the photodetector. If the light source 242 and the photodetector 248 were disposed side-by-side in the transverse plane along different diameters of the ingestible device 200, respectively, then the amount of emission light specularly reflected off the assay window 238 into the photodetector could be reduced, as described below.

### Example 2

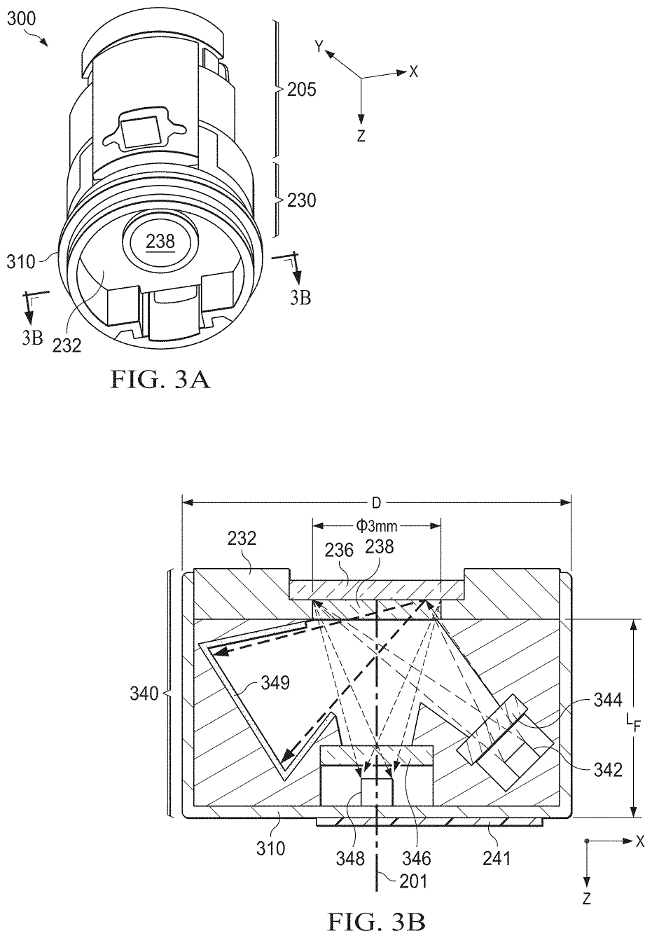

FIGS. 3A and 3B are cutaway diagrams showing different portions of an ingestible device 300 that includes a fluorometer 340. In addition to the fluorometer 340, the ingestible device 300 also includes a sample chamber 230 and a service system 205. The sample chamber 230 and the service system 205 were described above in connection with FIGS. 2A-2C. Housing 310 encloses the service system 205, the sample chamber 230 and the fluorometer 340 of the ingestible device 300. The sample chamber 230 is separated from the fluorometer 340 by a sample-chamber base 232 that is disposed in the (x,y)-plane, which is designed to allow both emission light and excitation light to pass between the fluorometer 240 and the sample chamber 230. The sample chamber 230 has an aperture covered by an assay window 238, as in the examples described above. The assay pad 236 is disposed adjacent to the sample-chamber side of the assay window 238 to hold a sample. In the example illustrated in FIGS. 3A-3B, the sample chamber 230 can have the dimensions as described above with respect to the ingestible device 200.

The fluorometer 340 includes a light source 342, an excitation filter 344 disposed between the light source and the assay window 238, a photodetector 348, and an emission filter 346 disposed between the assay window and the photodetector. Here, the light source 342, the excitation filter 344, the emission filter 346, and the photodetector 348 can be configured like their counterpart components of the fluorometer 240, as described above in connection with FIGS. 2A-2C. Moreover, note that the light source 342 and the photodetector 348 also are adjacent to the PCB 241 and are electrically coupled to respective electronic circuitry printed on the PCB. However, in the example illustrated in FIGS. 3A-3B, the light source 342 and the photodetector 348 of the fluorometer 340 are disposed side-by-side in a transverse plane, e.g., parallel to the (x,y)-plane, along different diameters of the ingestible device 300, respectively. For instance, the light source 342 can be disposed on a diameter parallel to the x-axis, and the photodetector 348 can be disposed on another diameter parallel to the y-axis. This relative orientation of the light source 342, the assay window 238 and the photodetector 348 causes excitation light emitted by the light source to specularly reflect off the assay window away from the photodetector. To minimize the amount of specularly reflected emission light that would be scattered by the housing 310 into the photodetector 348, the fluorometer 340 includes a beam dump 349 formed from material that absorbs the excitation light. The beam dump 349 is—(i) along the same diameter of the ingestible device 300 as the light source 342, and (ii) symmetrically relative to the optical axis 201 of the assay window 238—to capture the excitation light specularly reflected off the assay window.

Note that in the examples of fluorometer 200, 300 described above, the light source is spaced apart from the assay window 238, i.e., the excitation light is emitted remotely from the assay window, and thus from the sample held by the assay pad 236 on the sample-chamber side of the assay window. To increase the amount of excitation light that reaches the sample, excitation light can be delivered directly to the sample, or at least directly to the assay window 238, as described below.

### Example 3

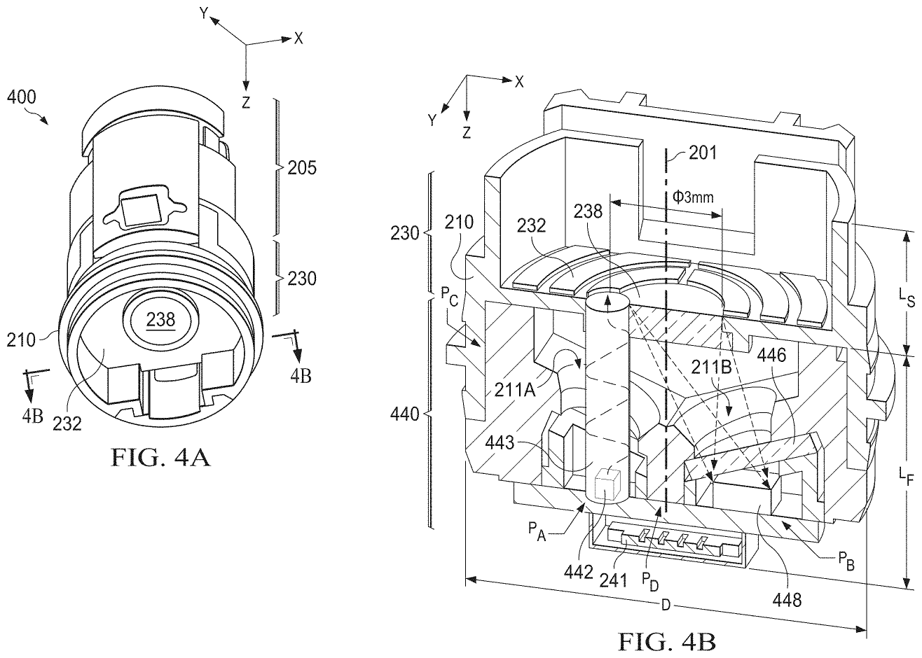

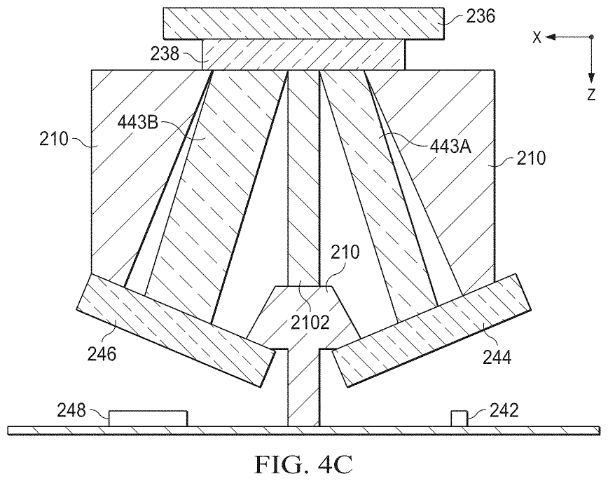

FIGS. 4A-4B are cutaway diagrams showing different portions of an ingestible device 400 that includes a fluorometer 440. In addition to the fluorometer 440, the ingestible device 400 also includes a sample chamber 230 and a service system 205. The sample chamber 230 and the service system 205 were described above in connection with FIGS. 2A-2C. Housing 210 encloses the service system 205, the sample chamber 230 and the fluorometer 440 of the ingestible device 400. The sample chamber 230 is separated from the fluorometer 440 by a sample-chamber base 232 that is disposed in the (x,y)-plane, which is designed to allow both emission light and excitation light to pass between the fluorometer 440 and the sample chamber 230. The sample chamber 230 has an aperture covered by an assay window 238, as in the examples described above. In the example illustrated in FIGS. 4A-4B, the sample chamber 230 and the fluorometer 440 can have the dimensions as described above with respect to the sample chamber and fluorometer of the ingestible device 200.

The fluorometer 440 includes a light source 442, a photodetector 448, and an emission filter 446 disposed between the assay window 238 and the photodetector. Here, the light source 442, the emission filter 446 and the photodetector 448 can be configured like their counterpart components of the fluorometer 240, as described above in connection with FIGS. 2A-2C. In the example illustrated in FIGS. 4A-4B, the light source 442 and the photodetector 448 also are adjacent to the PCB 241 and are electrically coupled to respective electronic circuitry printed on the PCB. In the example illustrated in FIGS. 4A-4B, the light source 442 and photodetector 448 are disposed side-by-side in a transverse plane of the ingestible device 200, e.g., parallel to the (x,y)-plane, at respective positions P_{A} and P_{B}.

In this example, the fluorometer 440 further includes a light guide 443 optically coupled at an input end with the light source 442. An output end of the light guide 443, opposing the input end, is disposed adjacent the assay window 238. In some implementations, the light guide 443 ends inside the fluorometer 440, so its output end is adjacent to the fluorometer side of the assay window 238. In other implementations, the light guide 443 traverses the sample-chamber base 232 and ends inside the sample chamber 230, so the output end of the light guide is adjacent to the sample-chamber side of the assay window 238. In this case, because the assay pad 236 holding the sample is disposed adjacent to the sample-chamber side of the assay window 238, the light guide 443 can end at the sample itself.

In some implementations, the light guide 443 can be implemented as a light pipe that extends, e.g., along the z-axis, from the light source 442 to the assay window 238 through a first aperture 211A of an internal surface of the housing 210. The light pipe can be configured as a multimode optical fiber, or as a fiber bundle/ribbon that includes two or more multimode optical fibers. In other implementations, the light guide can be implemented as a slab of transparent dielectric material having rectangular cross-section, for instance.

During operation, the light source 442 emits excitation light into the input end of the light guide 443. The excitation light is guided through the light guide 443 to its output end where it is provided to the sample. The excitation light provided to the sample through the light guide 443 excites the sample, so the sample emits emission light through the assay window 238 back into the fluorometer 440. A portion of the emission light is emitted towards the photodetector 448 positioned at P_{B} along a prevalent direction extending through a second aperture 211B of the internal surface of the housing 210, and then through the emission filter 446. The emission filter 446 is configured to block excitation light that reaches the emission filter after spurious scattering off internal housing of the fluorometer 440, and transmit emission light that reaches the emission filter from the sample, after propagating through the assay window 238 and the free space between the assay window and the emission filter. Emission light collected by the photodetector 448 after transmission through the emission filter 446 can be used to determine information about the sample held by the assay pad 236. By providing excitation light directly to the sample, more of the excitation light emitted by the light source 442 will be used to excite the sample, so less spurious excitation light will propagate to the photodetector 448, compared to amounts of spurious excitation light propagating to the photodetector for fluorometers 200 or 300.

The ratio of signal light to background light received by the photodetector 448 could be further increased if a second light guide were added to guide the emission light from the assay window 238 to the emission filter 446 without losing emission light through spurious scattering. FIG. 4C is a side view of a portion of yet another embodiment of the fluorometer 240 that includes, in addition to the components of the embodiment illustrated in FIG. 2R, a first light guide 443A and a second light guide 443B.

The first light guide 443A is optically coupled (i) at its input end to the excitation filter 244 to receive excitation light from the light source 242, and (ii) at its output end to the assay window 238 to provide the excitation light guided there through to the assay pad 236. The second light guide 443B is optically coupled (i) at its input end to the assay window 238 to receive emission light from the assay pad 236, and at its output end to the emission filter 246 to provide the emission light guided there through to the photodetector 248. In this example, the first and second light guides 443A, 443B are disposed within the first and second tunnel paths 2105A, 2105B. If the first and second light guides 443A, 443B were added to the embodiment of the fluorometer 240 illustrated in FIG. 2P, then the first and second light guides 443A, 443B would be disposed within the first and second enclosures 2101A, 2101B.

The first light guide 443A is configured like the light guide 443 to guide the excitation light through TIR. The second light guide 443B is configured similarly to the light guide 443 to guide the emission light through TIR.

In some implementations, the cross-section of each of the first and second light guides 443A, 443B is constant from its input end to its output end. For example, the cross-section anywhere between its input and output ends can be circular with the same radius. In other implementations, the cross-section of each of the first and second light guides 443A, 443B can vary from its input end to its output end. For example, the cross-section is circular between its input and output ends, but the cross-section's radius can increase from the input end towards the output end to output light with a divergence smaller than a divergence of input light. As another example, the cross-section is circular between its input and output ends, but the cross-section's radius can decrease from the input end towards the output end to output light with a divergence larger than a divergence of input light. As yet another example, a cross-section of the first light guide 443A can be circular at its input end adjacent to the excitation filter 244, and the cross-section can be shaped as a segment of a circle at its output end adjacent to the assay window 238. Here, a cross-section of the second light guide 443B can be can be shaped as a segment of a circle at its input end adjacent to the excitation filter 244, and the cross-section can be circular at its output end adjacent to the emission filter 246. By having cross-sections shaped as segments of a circle at the end of the light guides 443A, 443B adjacent to the assay window 238 ensures better light coupling in/out of the assay pad 236 on the opposite sides of the separator 2102 when compared to the case of circular cross-sections, as explained above in connection with FIGS. 2Qa-2Qc.

Referring again to the embodiment of the fluorometer 440 illustrated in FIG. 4B, in other implementations, the light source 442 can be disposed on a surface of the internal housing of the fluorometer 440 that is orthogonal to the (x,y)-plane, e.g., at position P_{C}, instead of being disposed at position P_{A} on the transverse surface parallel to the (x,y)-plane, as described above. A light guide having an input end coupled with the light source 442 disposed at position P_{C} is shorter than the light guide 443 having the input end coupled with the light source 442 disposed at position P_{A}, and is oriented radially within the fluorometer 440, over at least some portions of the light guide. In some implementations, loses in such a shorter light guide could be smaller than in the longer light guide 443.

In implementations when the light source 442 is disposed at position P_{C}, the photodetector 448 and its associated emission filter 446 can remain, in some cases, at position P_{B}, as described above. In other cases, the photodetector 448 and its associated emission filter 446 can be shifted on the transverse surface parallel to the (x,y)-plane at position P_{D}, which is centered on the assay window 238. In such case, the second aperture 211B would be shifted laterally to also be centered on the assay window 238. Because the position P_{D} is on the optical axis 201 of the assay window 238, a larger fraction of the emission light will reach the photodetector 448 positioned at P_{D} than when the photodetector is positioned at P_{B}.

Moreover, when the light source 442 is disposed at position P_{C} and the photodetector 448 is disposed at position P_{D}, a collection optic can be coupled to, or be part of, the assay mirror 238 to further improve collection efficiency of the emission light. Such an implementation of a fluorometer is described next.

### Example 4

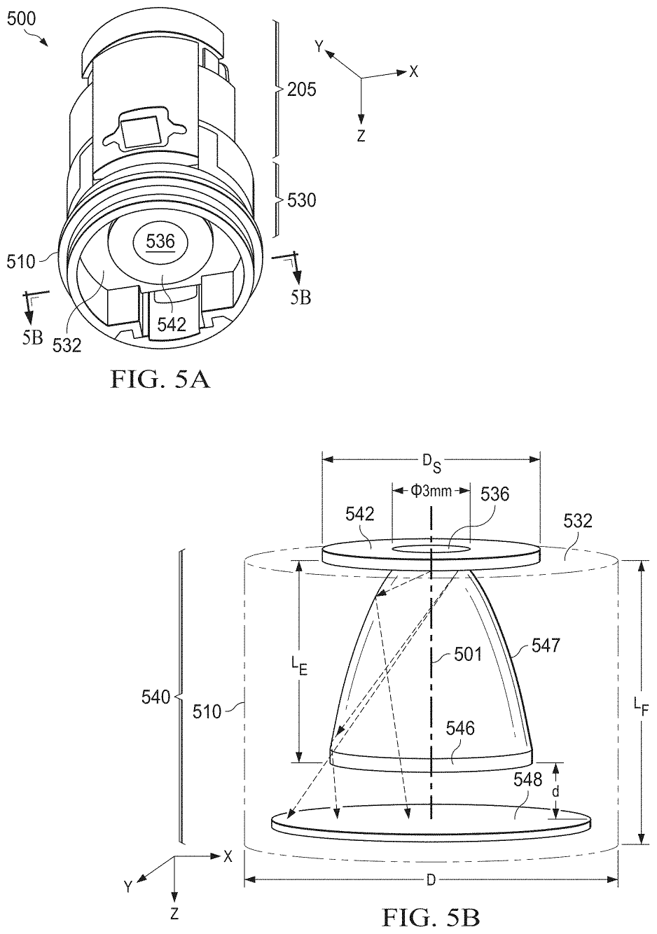

FIGS. 5A-5B are cutaway diagrams showing different portions of an ingestible device 500 that includes a fluorometer 540. In addition to the fluorometer 540, the ingestible device 500 also includes a sample chamber 530 and a service system 205. The service system 205 was described above in connection with FIG. 2A. Housing 510 encloses the service system 205, the sample chamber 530 and the fluorometer 540 of the ingestible device 500.

The sample chamber 530 is separated from the fluorometer 540 by a sample-chamber base 532 that is disposed in the (x,y)-plane. The sample-chamber base 532 has an aperture through which (at least a portion of) an assay pad 536 protrudes into the fluorometer 540. In the example shown in FIGS. 5A-5B, the aperture is circularly shaped, so the portion of the assay pad 536 protruding through the aperture is disk shaped. In other implementations, the aperture can be shaped like a polygon with three or more sides, so the portion of the assay pad 536 protruding through the aperture is prism shaped. In the example illustrated in FIGS. 5A-5B, the sample chamber 530 can have the dimensions as described above with respect to the ingestible device 200. In some embodiments, the fluorometer 540 has a longitudinal size L_{F}=6-10 mm, e.g., along the z-axis, and a transverse size D=8-13 mm, e.g., in the (x,y)-plane.

The fluorometer 540 includes a light source 542 having annular shape, e.g., shaped like a disk having a circular opening concentric with the disk perimeter. The annular-shaped light source 542 is configured to emit excitation light uniformly around the inner edge of the source. For this reason, the annular-shaped light source 542 is said to emit a ring of excitation light. In the example illustrated in FIGS. 5A-5B, the annular-shaped light source 542 is attached to the fluorometer side of the sample-chamber base 532 such that the opening of the light source matches the aperture of the sample-chamber base. In this manner, the inner edge of the annular-shaped light source 542 encircles the (portion of the) assay pad 536 protruding through the aperture of the sample-chamber base 532. During operation of the fluorometer 540, excitation light emitted by the annular-shaped light source 542 will be edge coupled into the assay pad 536 to excite the sample held in the assay pad. The excited sample emits emission light away from the assay pad 536.

The fluorometer 540 also includes a fluorescence-collection optic 547 coupled with the assay pad 536, a photodetector 548, and an emission filter 546 disposed between the fluorescence-collection optic and the photodetector. These components have a common optical axis 501. The emission filter 546 and the photodetector 548 can be configured like their counterpart components of the fluorometer 240, as described above in connection with FIGS. 2A-2C.

In the example illustrated in FIG. 5B, the fluorescence-collection optic 547 is configured as a solid compound parabolic concentrator (CPC). In some implementations, fluorescence-collection optics can include a lens (e.g., conventional, Fresnel, or gradient index lens), one or more mirrors, any number of variations of CPC (filled or unfilled; with circular or polygonal cross-sections in the (x,y)-plane) or a combination thereof. Referring again to FIG. 5B, the CPC 547 can be formed from a transparent dielectric material, e.g., glass or polymer. An input end of the CPC 547 is disposed adjacent to, and is optically coupled with, the fluorometer side of the assay pad 536, while an output end of the CPC is facing the photodetector 548. The CPC 547 can have a length between its input and output ends L_{E}=5-6 mm.

The emission light is emitted from the assay pad 536 along the z-axis over a large angular extent, e.g., in accordance with a Lambertian angular distribution. The input end of the CPC 547 receives the emission light from the assay pad 536. A side surface of the CPC 547 is shaped to concentrate the emission light from an input angular range having a given (large) divergence at its input end to an output angular range have smaller divergence as it exits the CPC through its output end. The shape of the CPC 547's side surface is designed to cause the emission light to propagate between the input and output ends of the CPC through total internal reflection (TIR). Nonetheless, rays of the emission light which enter the input end of the CPC 547 at too high angle will leak out through the CPC's side surface and will be absorbed and/or scattered by the inner housing of the fluorometer 540. To avoid such leakage, the CPC 547 can be configured as a “hollow” CPC for which the side surface is made from a reflective material, e.g., Al, Ag, etc. A combination of (i) the shape of the CPC 547's side surface, (ii) the length L_{E} of the CPC, and (iii) a distance d between the output end and the photodetector 548 is configured to cause all the emission light output by the CPC to fall within the area of the photodetector. The distance between the output end and the photodetector 548 can be d=0.5-2 mm.

The emission filter 546 can be attached to the output end of the CPC 547 to block excitation light that has not been used to excite the sample in the assay pad 536 and propagated between the input and output ends of the CPC. Additionally, the emission filter 546 transmits emission light that reaches the output end of the CPC 547 from the sample, after it propagated between the input and output ends of the CPC. Note that the shape of the CPC 547's side surface is configured to cause a divergence of the emission light within the output angular range to be smaller than a maximum acceptable angle-of-arrival for the emission filter 546 to reject excitation light.

Emission light collected by the photodetector 548 after transmission through the emission filter 546 can be used to determine information about the sample held by the assay pad 536. Because the CPC 547 causes that most of the emission light emitted by the sample in the assay pad 536 to be collected by the photodetector 548, the fluorometer 540 is expected to be more sensitive, e.g., have a higher ratio of signal light to background light, than fluorometers 240, 340 and 440 in which only a fraction of the emission light is collected by respective photodetectors 248, 348, and 448, which are operated without the benefit of a CPC.

Note that use of the above combination of CPC 547 and emission filter 546 ensures that no excitation light reaches the photodetector 548, effectively removing the scattered excitation light as a source of background light for the detection of emission light.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Although certain embodiments have been described, other embodiments are possible. Features described for a given embodiment (e.g., shown in a given figure or subset of figures) can be combined with features described with one or more different embodiments (e.g., shown in one or more different figures).

Similarly, while operations are depicted in the drawings and recited in the claims in a particular order, this should not be understood as requiring that such operations be performed in the particular order shown or in sequential order, or that all illustrated operations be performed, to achieve desirable results. For example, the actions recited in the claims can be performed in a different order and still achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims.

What is claimed is:

1. An ingestible device, comprising:
a sample chamber configured to hold a sample, the sample chamber comprising a base having an aperture;
a fluorometer separated from the sample chamber by the sample chamber base, the fluorometer comprising
a light source configured to emit excitation light,
a light guide configured to guide the excitation light from the light source to the sample,
a photodetector configured to detect emission light generated via the interaction of the excitation light with the sample, wherein the photodetector is configured to receive the emission light from the sample through the aperture, and
an emission filter between the aperture and the photodetector, the emission filter configured to (i) transmit the emission light incident thereon, and (ii) block excitation light incident thereon; and
a housing houses the sample chamber and the fluorometer.

2. The ingestible device of claim 1, wherein the light guide comprises one or more optical fibers.

3. The ingestible device of claim 2, wherein the light guide comprises an optical-fiber bundle.

4. The ingestible device of claim 1, wherein the sample chamber comprises an assay pad disposed adjacent to the aperture of the sample chamber base and configured to hold the sample.

5. The ingestible device of claim 4, wherein the light guide has an input end coupled with the light source and an output end coupled with the assay pad, the light guide configured to (i) receive the excitation light at the input end, (ii) guide the excitation light from the input end to the output end, and (iii) deliver the excitation light through the output end to the assay pad.

6. The ingestible device of claim 5, wherein the light guide is configured such that the output end is coupled to a surface of the assay pad facing the sample chamber to deliver the excitation light towards the fluorometer.

7. The ingestible device of claim 5, wherein the light guide is configured such that the output end is coupled to a surface of the assay pad facing the fluorometer to deliver the excitation light towards the sample chamber.

8. The ingestible device of claim 1, wherein the light source is disposed on an inner surface of the housing that is (i) part of the fluorometer, and (ii) oriented parallel to the sample-chamber base.

9. The ingestible device of claim 8, wherein the light source is disposed on the same inner surface of the housing as the photodetector.

10. The ingestible device of claim 1, wherein the light source is disposed on an inner surface of the housing that is (i) part of the fluorometer, and (ii) oriented orthogonal to the sample-chamber base.

11. The ingestible device of claim 1, wherein the photodetector is disposed on an inner surface of the housing that is part of the fluorometer at a position where an optical axis of the aperture intersects the inner surface of the housing.

12. The ingestible device of claim 1, wherein inner surfaces of the housing that are part of the fluorometer comprise a light-absorbing material configured to absorb excitation light incident thereon.

13. An ingestible device, comprising:
a sample chamber having a base and comprising an assay pad configured to hold a sample, wherein the sample chamber base has an aperture, and the assay pad is disposed adjacent to the aperture;
a fluorometer separated from the sample chamber by the sample-chamber base, the fluorometer comprising
an annular-shaped light source having an inner edge, the annular-shaped light source configured to emit excitation light at its inner edge, the annular-shaped light source being attached to a fluorometer-side surface of the sample-chamber base to deliver the excitation light to a portion of the assay pad that (i) protrudes through the aperture and (ii) is encircled by the inner edge of the annular-shaped light source,
a photodetector configured to detect emission light generated via the interaction of the excitation light with the sample,
a fluorescence-collection optic having an input end coupled to the assay pad and an output end coupled with the photodetector, the fluorescence-collection optic configured to (i) receive at the input end emission light emitted by the sample with a first divergence, and (ii) provide, at the output end to the photodetector, the emitted light with a second divergence smaller than the first divergence, and
an emission filter between the output end of the fluorescence-collection optic and the photodetector, the emission filter configured to (i) transmit the emission light incident thereon, and (ii) block the excitation light incident thereon; and
a housing that houses the sample chamber and the fluorometer.

14. The ingestible device of claim 13, wherein the fluorescence-collection optic is configured as a compound parabolic concentrator (CPC).

15. The ingestible device of claim 14, wherein
the CPC is a solid CPC comprising a dielectric material that is transparent to emission light, and
a side surface of the CPC is shaped to reflect the emission light received at the input end through total internal reflection (TIR).

16. The ingestible device of claim 14, wherein the CPC is a hollow CPC comprising a reflective material.

17. The ingestible device of claim 13, wherein
the emission filter is coupled at the output end of the fluorescence-collection optic, and
the photodetector is spaced apart from the emission filter.

18. The ingestible device of claim 13, wherein the photodetector is spaced apart from the emission filter by a separation distance in a range of 0-1.5 mm.

19. The ingestible device of claim 13, wherein the fluorescence-collection optic has a longitudinal size in a range of 5-6 mm.

20. An ingestible device, comprising:
a sample chamber configured to hold a sample, the sample chamber having a base with an aperture;
a fluorometer separated from the sample chamber by the sample-chamber base, the fluorometer comprising
a light source configured to emit excitation light,
a photodetector configured to detect emission light generated via the interaction of the excitation light with sample, wherein the photodetector is configured to receive the emission light from the sample through the aperture, and
an emission filter between the aperture and the photodetector, the emission filter configured to (i) transmit the emission light incident thereon, and (ii) block the excitation light incident thereon; and
a housing that houses the sample chamber and the fluorometer, wherein inner surfaces of the housing that are part of the fluorometer comprise a light-absorbing material configured to absorb excitation light incident thereon.

21-61. (canceled)

62. A system comprising:
the ingestible device of claim 20; and
a hardware processor configured to produce information about the sample based on characteristics of the detected emission light.
